SEQUENCE LISTING (3 sequences)

SEQ ID NO: 1 - protein, 2332 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 2 - protein, 19 aa, synthetic construct
MQIELSTCFFLCLLRFCFS

SEQ ID NO: 3 - protein, 14 aa, synthetic construct
SFSQNPPVLKRHQR

(19) United States
(12) Patent Application Publication
(10) Pub. No.: US 2025/0250322 A1
(43) Pub. Date: Aug. 7, 2025

(54) COMPOSITIONS AND METHODS FOR MODULATING FACTOR VIII FUNCTION

(71) Applicant: The Children's Hospital of Philadelphia, Philadelphia, PA (US)

(72) Inventor: Lindsey A. George, Haddonfield, NJ (US)

(21) Appl. No.: 18/856,176

(22) PCT Filed: Apr. 25, 2023
(86) PCT No.: PCT/US2023/066152

§ 371 Date: Oct. 11, 2024

Related U.S. Application Data
(60) Provisional application No. 63/334,289, filed on Apr. 25, 2022.

(51) Int. Cl. C07K 14/755; A61K 38/37; A61P 7/04

(52) U.S. Cl. CPC C07K 14/755; A61K 38/37; A61P 7/04

(57) ABSTRACT

Factor VIII variants and methods of use thereof are disclosed.

This application claims priority under 35 U.S.C. § 119 (e) to U.S. Provisional Patent Application No. 63/334,289, filed Apr. 25, 2022. The foregoing application is incorporated by reference herein.

This invention was made with government support under Grant Number NHLBI K08 HL 146991-01 awarded by the National Institutes of Health. The government has certain rights in the invention.

### FIELD OF THE INVENTION

The present invention relates to the fields of medicine and hematology. More specifically, the invention provides novel Factor VIII variants and methods of using the same to modulate the coagulation cascade in patients in need thereof.

### BACKGROUND OF THE INVENTION

Several publications and patent documents are cited throughout the specification in order to describe the state of the art to which this invention pertains. Each of these citations is incorporated herein by reference as though set forth in full.

Coagulation factor VIII (FVIII) circulates in blood tightly bound to its carrier protein, von Willebrand factor (vWF) (Eaton, et al. (1986) Biochemistry 25 (2): 505-512; Vehar, et al. (1984) Nature 312 (5992): 337-342; Lollar, et al. (1988) J. Biol. Chem., 263 (21): 10451-10455). Proteolytic processing by thrombin liberates FVIII from vWF and produces the active cofactor species (FVIIIa), which is a heterotrimer comprised of an A2-domain weakly associated with the metal ion-stabilized A1/A3-C1-C2 heterodimer (Vehar, et al. (1984) Nature (1984) 312 (5992): 337-342; Fay, et al. (1992) J. Biol. Chem., 267 (19): 13246-13250). Factor VIIIa associates with activated FIX (FIXa) on anionic phospholipid surfaces forming the intrinsic Xase enzyme complex, one of two enzymes that activates FX (Eaton, et al. (1986) Biochemistry 25 (2): 505-512; Hill-Eubanks, et al. (1990) J. Biol. Chem., 265 (29): 17854-17858; Lenting, et al. (1994) J. Biol. Chem., 269 (10): 7150-7155; Venkateswarlu, D. (2014) Biochem. Biophys. Res. Comm., 452 (3): 408-414; Kolkman, et al. (1999) Biochem J., 339 (Pt 2): 217-221; Fay, et al. (1998) J. Biol. Chem., 273 (30): 19049-19054; Kolkman, et al. (1999) 274 (41): 29087-29093; Kolkman, et al. (2000) Biochemistry 39 (25): 7398-7405). Deficiency or dysfunction of FVIII results in hemophilia A (HA), highlighting the importance of FVIIIa cofactor function. Downregulation of intrinsic Xase function is achieved through inhibition of FIXa by antithrombin and possibly protein S (PS), and FVIIIa inactivation by spontaneous A2-domain dissociation or proteolytic cleavage at Arg336 and Arg562 by activated protein C (APC) (Lollar, et al. (1991) J. Biol. Chem., 266 (19): 12481-12486; Hultin, et al. (1981) Blood 57 (3): 476-482; Lollar, et al. (1984) Blood 63 (6): 1303-1308; Lollar, et al. (1990) J. Biol. Chem., 265 (3): 1688-1692; Walker, et al. (1987) Arch. Biochem. Biophys., 252 (1): 322-328; Plautz, et al. (2018) Arterioscler. Thromb. Vasc. Biol., 38 (4): 816-828; Fay, et al. (1991) J. Biol. Chem., 266 (30): 20139-20145). Because FVIIIa has such a profound effect (103-106-fold) on increasing FIXa function, its inactivation is important for regulating intrinsic Xase function (van Dieijen, et al. (1981) J. Biol. Chem., 256 (7): 3433-3442; Mertens, et al. (1984) Biochem. J., 223 (3): 599-605).

Following activation by thrombin, FVIIIa loses activity in minutes due to spontaneous A2-domain dissociation (Lollar, et al. (1991) J. Biol. Chem., 266 (19): 12481-12486; Hultin, et al. (1981) Blood 57 (3): 476-482; Lollar, et al. (1984) Blood 63 (6): 1303-1308; Lollar, et al. (1990) J. Biol. Chem., 265 (3): 1688-1692; Lu, et al. (1996) Blood 87 (11): 4708-4717; Fay, et al. (1991) J. Biol. Chem., 266 (14): 8957-8962). The physiologic relevance of this mechanism is exemplified by a number of mild HA mutations that diminish A2 affinity within the FVIIIa heterotrimer (McGinniss, et al. (1993) Genomics 15 (2): 392-398; Duncan, et al. (1994) Br. J. Haematol., 87 (4): 846-848; Rudzki, et al. (1996) Br. J. Haematol., 94 (2): 400-406; Hakeos, et al. (2002) Thromb. Haemost., 88 (5): 781-787; Pipe, et al. (2001) Blood 97 (3): 685-691; Pipe, et al. (1999) Blood 93 (1): 176-183). The presumed importance of A2-domain dissociation in regulating FVIIIa function has been exploited to successfully bioengineer variants with enhanced inter-domain interactions that confer improved hemostatic function (Leong, et al. (2015) Blood 125 (2): 392-398; Wakabayashi, et al. (2008) Blood 112 (7): 2761-2769; Gale, et al. (2003) J. Thromb. Haemostasis 1 (9): 1966-1971; Gale, et al. (2008) J. Biol. Chem., 283 (24): 16355-16362). Collectively, available biochemical, clinical, and in vivo data support A2-domain dissociation as an important mechanism regulating FVIIIa function. In contrast, previous biochemical studies show that FVIIIa inactivation by APC occurs over hours (Fay, et al. (1991) J. Biol. Chem., 266 (30): 20139-20145; Lu, et al. (1996) Blood 87 (11): 4708-4717). The faster rate of A2-dissociation compared to APC cleavage has implicated the former as the predominant mechanism of FVIIIa inactivation (Lollar, et al. (1991) J. Biol. Chem., 266 (19): 12481-12486; Hultin, et al. (1981) Blood 57 (3): 476-482; Lollar, et al. (1984) Blood 63 (6): 1303-1308; Lollar, et al. (1990) J. Biol. Chem., 265 (3): 1688-1692; Lu, et al. (1996) Blood 87 (11): 4708-4717; Fay, et al. (1991) J. Biol. Chem., 266 (14): 8957-8962). Consistent with this understanding, there is no described clinical phenotype associated with altered APC cleavage of FVIII/FVIIIa (Bezemer, et al. (2008) JAMA 299 (11): 1306-1314; EAHAD F8 Gene Variant Database). This is in contrast to FV, which is similar to FVIII, where APC resistance (FV-Leiden, Arg506Gln) imparts a 50- to 100-fold and 5- to 10-fold increased venous thrombosis risk in the homozygous or heterozygous state, respectively, and is the most common inherited thrombophilia (Bertina, et al. (1994) Nature 369 (6475): 64-67; Zoller, et al. (1994) Lancet 343 (8912): 1536-1538; Zoller, et al. (1994) J. Clin. Invest., 94 (6): 2521-2524; Juul, et al. (2002) Blood 100 (1): 3-10; Suzuki, et al. (1983) J. Biol. Chem., 258:1914-1920).

As explained above, mutations in Factor VIII (FVIII) can lead to severe bleeding disorders and are associated with hemophilia A. Defective FVIII or a lack of FVIII activity results in an inability to effectively form clots. To date, only 20% of patients with hemophilia A worldwide receive regular treatment with FVIII replacement therapy due its high cost. Typically, FVIII therapy is plasma-derived or recombinantly produced. Gene therapy for hemophilia A based on AAV vectors is promising. Thus, generating enhanced function FVIII variants would benefit the treatment of hemophilia. Therefore, there is an obvious need for FVIII molecules with improved biological properties.

### SUMMARY OF THE INVENTION

In accordance with the present invention, compositions and methods for the modulation of hemostasis in patients in need thereof are provided. More specifically, Factor VIII (FVIII) variants which modulate (e.g., increase) hemostasis (e.g., increase clot formation) are provided. In certain embodiments, the Factor VIII variant comprises at least one mutation at positions 336, 519, 562, and 665. In certain embodiments, the Arg at position 336 and/or 562 is substituted with Gln. In certain embodiments, the Asp or Glu at positions 519 and/or 665 is substituted with Val. Compositions comprising at least one FVIII variant of the instant invention and at least one pharmaceutically acceptable carrier are also provided. Nucleic acid molecules encoding the FVIII variants of the invention are also disclosed as are methods of use thereof. Compositions comprising at least one FVIII variant encoding nucleic acid molecules of the instant invention and at least one pharmaceutically acceptable carrier are also provided. Another aspect of the invention includes host cells expressing the FVIII variants described herein. Methods for isolating and purifying the FVIII variants are also disclosed.

Pharmaceutical compositions comprising the FVIII variants and/or FVIII variant encoding nucleic acid molecules of the invention in a carrier are also provided. The invention also includes methods for the treatment of a hemostasis related disorder in a patient in need thereof. In certain embodiments, the methods comprise administration of a therapeutically effective amount of the FVIII variant and/or FVIII variant encoding nucleic acid molecules, particularly within a pharmaceutical composition. In certain embodiments, the method comprises editing the FVIII gene. Such methods have efficacy in the treatment of disorders where a pro-coagulant is needed and include, without limitation, hemophilia, particularly hemophilia A.

### BRIEF DESCRIPTION OF THE DRAWINGS

FIG. 1A provides an amino acid sequence of FVIII (SEQ ID NO: 1). The amino acids at positions 336, 519, 562, and 665 are bolded and underlined. The B domain is also indicated with italics and bolding. The thrombin cleavage site arginines at 372, 740, and 1689 are indicated by italics and underlining. The provided amino acid sequence lacks the 19 amino acid signal peptide at the N-terminus (MQIELSTCFFLCLLRFCFS (SEQ ID NO: 2)). FIG. 1B provides a schematic of the FVIII domain structure with thrombin (IIa) cleavage sites and mutations noted. FIG. 1C provides schematic representations of certain FVIII proteins. SQ sequence is SFSQNPPVLKRHQR (SEQ ID NO: 3). FIG. 1D provides an image of an SDS-PAGE of purified species (2 μg) incubated with or without 100 nM thrombin in the presence of 4 μM PCPS for 20 minutes. FIG. 1E provides an image of a Western blot analysis of 20 nM FVIII incubated with or without 6 nM APC in the presence of 20 μM PCPS for 30 minutes.

FIG. 2A provides a graph of the specific activities of FVIII-SQ (B-domain deleted (BDD) FVIII, also referred to as WT), FVIII-QQ (R336Q/R562Q), FVIII-VV (D519V/E665V), and FVIII-QQVV (R336Q/R562Q/D519V/E665V). FIG. 2B provides a graph of peak thrombin generation by FVIII-SQ, FVIII-QQ, FVIII-VV, and FVIII-QQVV. FIG. 2C provides a graph of FVIIIa activity of FVIIIa-SQ, FVIIIa-QQ, FVIIIa-VV, and FVIIIa-QQVV over time. Here, the loss of FVIIIa activity denotes A2-domain dissociation. FIG. 2D provides a graph of FXa generation with FVIIIa-SQ, FVIIIa-QQ, FVIIIa-VV, and FVIIIa-QQVV. Km and Vmax values for each protein are also provided.

FIGS. 3A-3F show that the fractional saturation of FVIIIa with FIXa impacts the rate of FVIIIa inactivation. FIGS. 3A, 3C, and 3E represent the change in FVIIIa activity over a time course of 30 minutes at different levels of FVIIIa-FIXa saturation without APC and PS. FIGS. 3B, 3D, and 3F represent the same conditions with APC and PS. FIGS. 3A and 3B are 100% FVIIIa-FIXa bound; FIGS. 3C and 3D are 25% FVIIIa-FIXa bound; and FIGS. 3E and 3F are 0% FVIIIa-FIXa bound. FVIII-WT: black circles, FVIII-QQ: grey squares, FVIII-VV: grey triangles, and FVIII-QQVV: grey octagons. Graphs are representative of three independent experiments.

FIGS. 4A-4F show that assembly into the intrinsic Xase complex protects FVIIIa from APC cleavage. FIGS. 4A, 4C, and 4E represent the change in FVIIIa activity over a time course of 30 minutes at different levels of FVIIIa-FIXa saturation without APC and PS. FIGS. 4B, 4D, and 4F represent the same conditions with APC and PS. FIGS. 4A and 4B are 100% FVIIIa-FIXa bound; FIGS. 4C and 4D are 25% FVIIIa-FIXa bound; and FIGS. 4E and 4F are 0% FVIIIa-FIXa bound. FVIII-WT: black circles, FVIII-QQ: grey squares, FVIII-VV: grey triangles, and FVIII-QQVV: grey octagons. Graphs are representative of three independent experiments.

### DETAILED DESCRIPTION OF THE INVENTION

Hemophilia A (HA) and hemophilia B (HB) are X-linked bleeding disorders due to inheritable deficiencies in either coagulation factor VIII (FVIII) or factor IX (FIX), respectively (Peyvandi, et al., Lancet (2016) 388:187-197; Konkle, et al., Hemophilia A in GeneReviews, Adam, et al., eds., University of Washington (1993)). The bleeding phenotype is generally related to the residual factor activity: people with severe disease (factor activity<1% normal) have frequent spontaneous bleeds; people with moderate disease (factor activity 1%-5% normal) rarely have spontaneous bleeds, but bleed with minor trauma; and people with mild disease (factor activity 5%-40% normal) bleed during invasive procedures or trauma. Given this well-defined relationship between factor activity and bleeding phenotype, HA and HB are attractive targets for protein infusion or gene therapy as small increases in factor levels are expected to have a meaningful clinical impact.

As explained above, Factor VIII is central for coagulation activity and mutations in the FVIII gene result in hemophilia A, the most common form of hemophilia. Herein, specific changes in the amino acid sequence of FVIII are shown to be associated with enhanced protein resistance to proteolytic inactivation. Thus, the instant invention provides rationally designed amino acid residue modifications which provide unexpectedly superior variants.

There are two mechanisms contributing to FVIIIa inactivation: 1) rapid A2-domain dissociation and 2) APC cleavage. A2 dissociation is widely thought to be the predominant mechanism of FVIIIa regulation. FVIII mutants resistant to A2-domain dissociation are described (e.g., FVIII-VV (D519V/E665V); Wakabayashi et al. (2009) J Thromb Haemost., 7 (3): 438-444; Leong, et al. (2015) Blood 125 (2): 392-398; U.S. Patent Application Publication No. 2013/0085110, incorporated by reference herein). FVIII-VV demonstrates greater specific activity relative to FVIII-WT. Moreover, a FVIII variant resistant to APC cleavage (FVIII-QQ (R336Q/R562Q); PCT/US2020/063551; incorporated by reference herein) has also demonstrated enhanced hemostatic benefit using recombinant protein. Herein, it is demonstrated that combining mutations that inhibit APC cleavage and diminish A2-domain dissociation produces synergistic enhanced FVIII hemostatic function (e.g., for HA therapeutic benefit). Specifically, FVIII-QQVV (R336Q/R562Q/D519V/E665V) was generated, which is a protein resistant to APC cleavage and A2 dissociation, thereby inhibiting both mechanisms of FVIIIa regulation. As explained herein, FVIII-QQVV demonstrated unexpectedly superior properties compared to FVIII-QQ and/or FVIII-VV.

Full-length FVIII is a large, 280-kDa protein primarily expressed in liver sinusoidal endothelial cells (LSECs), as well as extra-hepatic endothelial cells (Fahs, et al., Blood (2014) 123:3706-3713; Everett, et al., Blood (2014) 123:3697-3705). FVIII predominantly circulates as a heterodimer of a heavy chain and a light chain bound through noncovalent metal-dependent interactions (Lenting, et al., Blood (1998) 92:3983-3996). Factor VIII comprises several domains and is 2332 amino acids in length (mature without signal peptide). Generally, the domains are referred to as A1-A2-B-A3-C1-C2. FVIII is translated as a single-peptide chain (single chain) with the domain structure of A1-α1-A2-α2-B-α3-A3-C1-C2. Proteolytic cleavage of FVIII at R-1313 and/or R-1648 by the trans-Golgi protease furin results in heterodimer formation. The FVIII heavy chain (A1-α1-A2-α2-B) and light chain (α3-A3-C1-C2) remain associated through non-covalent metal-ion-dependent interactions occurring between the A1 and A3 domains. Initially, FVIII is in an inactive form bound to von Willebrand factor (vWF). FVIII is activated by cleavage by thrombin (Factor IIa) and release of the B domain. The activated form of FVIII (FVIIIa) separates from vWF and interacts with coagulation factor Factor IXa-leading to the formation of a blood clot via a coagulation cascade. During coagulation, FVIII single chain or heterodimer is activated to its heterotrimeric cofactor form by cleavage by thrombin at R-372, R-740, and R-1689. A2 remains associated with A1-α1 via non-covalent interactions. Inactivation of FVIIIa occurs via spontaneous A2 dissociation and/or proteolytic cleavage, primarily by activated protein C, at R-336 and R-562.

The B domain comprises 40% of the protein (908 amino acids) and is not required for the protein procoagulant activity (Brinkhous, et al., Proc. Natl. Acad. Sci. (1985) 82:8752-8756). The most common B-domain deleted (BDD) FVIII comprises 14 original amino acid residues (SFSQNPPVLKRHQR (SEQ ID NO: 3)) as a linker (Lind, et al. (1995) Eur. J. Biochem., 232 (1): 19-27). This BDD FVIII is typically referred to as BDD-SQ or hFVIII-SQ. Short peptide linkers (e.g., 25 or fewer amino acids, 20 or fewer amino acids, 15 or fewer amino acids, or 10 or fewer amino acids) substituted for the B-domain can be used in FVIII variants (Lind, et al. (1995) Eur. J. Biochem., 232 (1): 19-27; Pittman, et al., Blood (1993) 81:2925-2935; Toole, et al., Proc. Natl. Acad. Sci. (1986) 83:5939-5942). In a particular embodiment, the peptide linker comprises a basic amino acid (e.g., Arg, His, or Lys) at position-1 and -4 to Glu1649. This BDD FVIII form is commonly used to produce recombinant BDD-FVIII (˜ 4.4 Kb) as well for gene therapy (Berntorp, E., Semin. Hematol. (2001) 38 (2 Suppl 4): 1-3; Gouw, et al., N. Engl. J. Med. (2013) 368:231-239; Xi, et al., J. Thromb. Haemost. (2013) 11:1655-1662; Recht, et al., Haemophilia (2009) 15:869-880; Sabatino, et al., Mol. Ther. (2011) 19:442-449; Scallan, et al., Blood (2003) 102:2031-2037). As noted above, gene therapy using AAV vectors can only use shortened FVIII molecules such as a BDD-FVIII due to the limited packaging capacity of the AAV (4.7 Kb) and other vector systems (Lind, et al. (1995) Eur. J. Biochem., 232 (1): 19-27). U.S. Pat. No. 8,816,054, incorporated by reference herein, also provides BDD FVIII molecules with linkers of different lengths and sequences.

FVIIIa is a cofactor for FIXa within the intrinsic Xase complex which functions to generate FXa, leading to the propagation of the coagulation cascade. As stated herein, FVIIIa inactivation is due to 1) spontaneous A2 dissociation or 2) activated Protein C (APC) proteolytic cleavage (e.g., cleavage of A2 into A2N and A2C). Biochemical and clinical data support the importance of A2 dissociation. Indeed, 90% of FVIIIa activity is lost after 5 minutes in a purified system (Lollar, et al. (1991) J. Biol. Chem., 266:12481-12486). Further, clinical data shows that ⅓ of patients with mild hemophilia have mutations that result in enhanced A2 dissociation. With regard to cleavage, APC cleavage results in the loss of 90% of FVIII activity after 4 hours in a purified system (Lu et al. (1996) Blood 87 (11): 4708-17). Unlike alterations in A2 dissociation, no known clinical phenotype is associated with altered APC cleavage.

In accordance with the instant invention, novel Factor VIII variants are provided. The instant invention encompasses FVIII variants including FVIIIa variants and FVIII prepeptide variants. For simplicity, the variants are generally described throughout the application in the context of FVIII. However, the invention contemplates and encompasses Factor FVIIIa and FVIII prepeptide molecules as well as Factor VIII domain(s) (e.g., A1 and/or A2 domain) with amino acid substitutions as described. In a particular embodiment, the FVIII variants are B-domain deleted (BDD) FVIII (optionally comprising a linker (e.g., an amino acid linker) in place of the B-domain). In a particular embodiment, the FVIII variants comprise A1-α1-A2-α2-B-α3-A3-C1-C₂. In a particular embodiment, the FVIII variants comprise A1-α1-A2-α2-α3-A3-C1-C₂. In a particular embodiment, the FVIII variants comprise A1-α1-A2-α2-A3-C1-C₂. In a particular embodiment, the FVIII variants comprise a light chain and a heavy chain.

As demonstrated herein, the FVIII variants of the instant invention possess greater resistance to APC cleavage than WT FVIII and greater resistance to A2 domain dissociation than WT FVIII. Moreover, it is also demonstrated herein that the FVIII variants of the instant invention have an unexpectedly superior hemostatic effect.

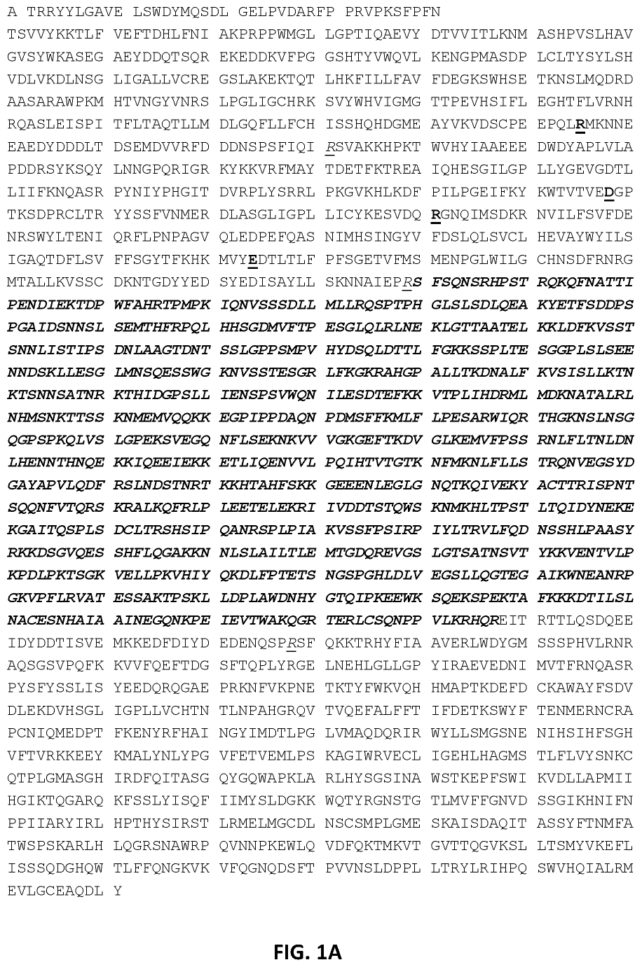

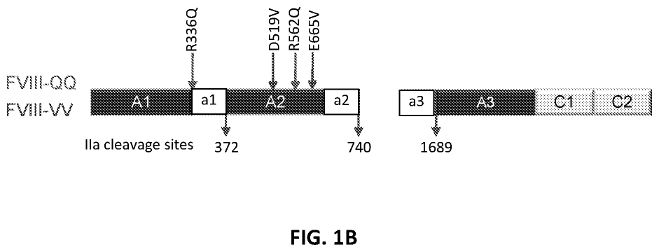

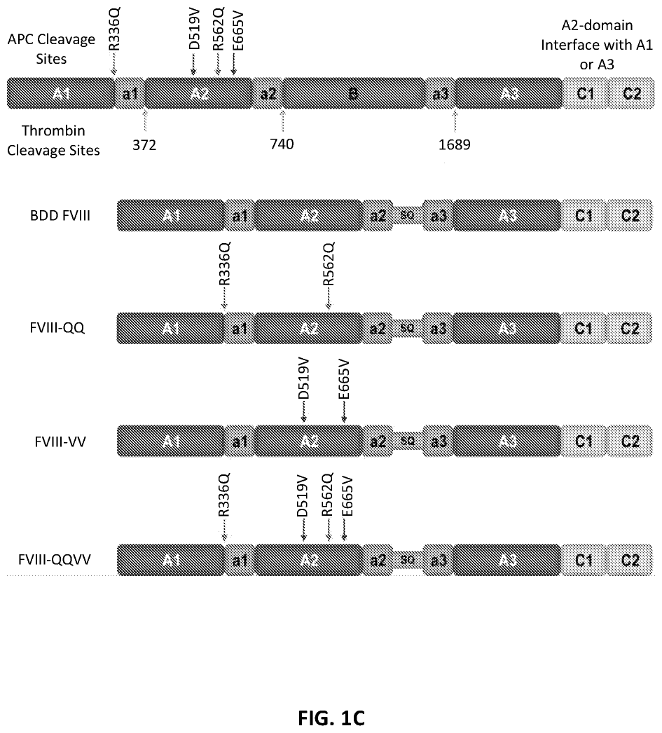

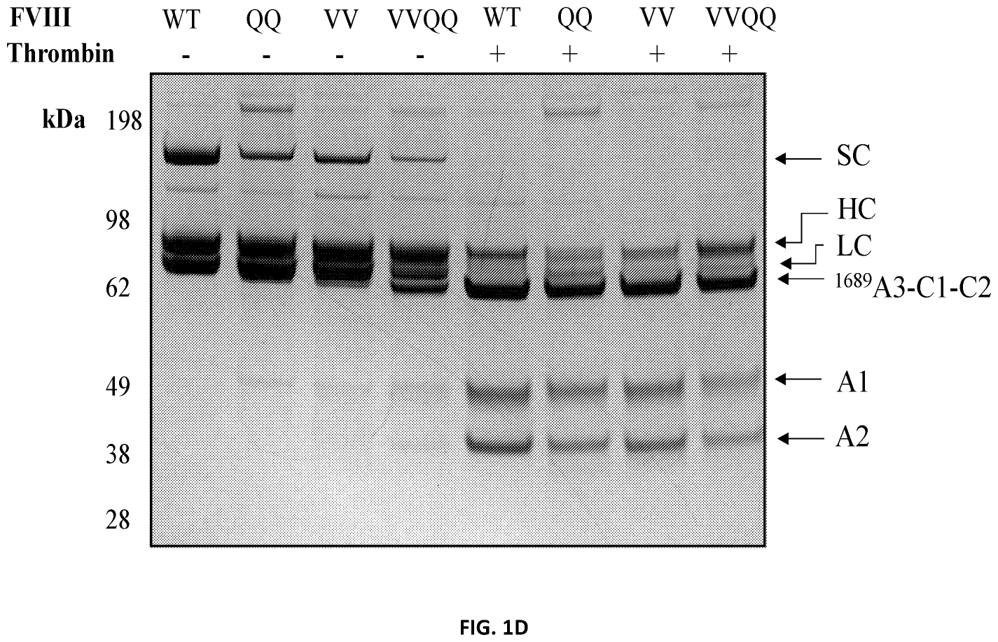

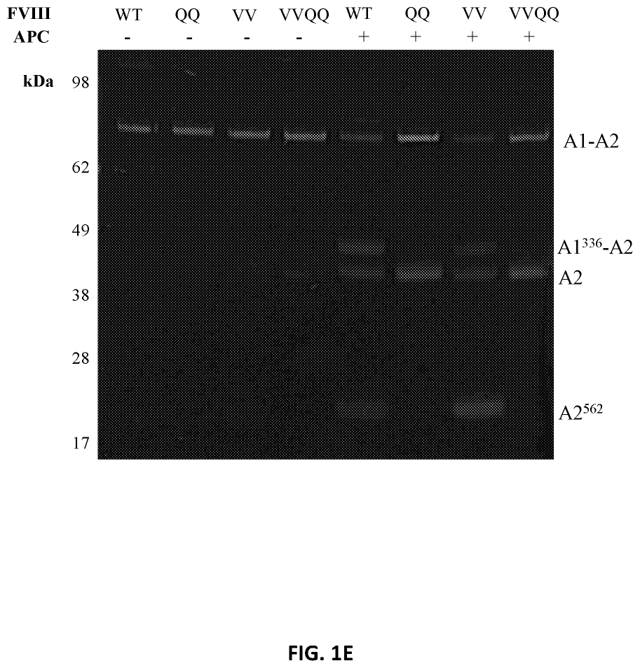

The FVIII variants of the instant invention can be from any mammalian species. In a particular embodiment, the FVIII variant is human. Gene ID: 2157 and GenBank Accession Nos. NM_000132.3 and NP_000123.1 provide examples of the amino acid and nucleotide sequences of wild-type human FVIII (particularly the prepeptide comprising the signal peptide). FIG. 1 provides SEQ ID NO: 1, which is an example of the amino acid sequence of human FVIII. SEQ ID NO: 1 lacks the 19 amino acid signal peptide at its N-terminus (MQIELSTCFFLCLLRFCFS (SEQ ID NO: 2)). Nucleic acid molecules which encode Factor FVIII variants can be readily determined from the provided amino acid sequences as well as the provided GenBank Accession Nos.

The Factor VIII variants of the instant invention may comprise a mutation(s) which provides APC resistance and a mutation(s) which reduces A2 domain dissociation. The Factor VIII variants of the instant invention may comprise at least one mutation at position 336, 519, 562, and/or 665. In certain embodiments, the Factor VIII variant comprises at least one mutation at position 336, 519, 562, and 665. In certain embodiments, the Factor VIII variant comprises R336Q, D519V, R562Q, and E665V. In certain embodiments, the Factor VIII variant further comprises a mutation at position 1984 (e.g., E1984X, wherein X is an amino acid other then Glu).

In certain embodiments, the Factor VIII variants comprise a mutation at position 336. In a particular embodiment, the Arg (R) at position 336 is not substituted with Lys (K). In a particular embodiment, the Arg at position 336 is substituted with Asp (D), Glu (E), Asn (N), or Gln (Q). In a particular embodiment, the Arg at position 336 is substituted with Asn (N) or Gln (Q). In a particular embodiment, the Arg at position 336 is substituted with Gln (Q).

In certain embodiments, the Factor VIII variants comprise a mutation at position 562. In a particular embodiment, the Arg (R) at position 562 is not substituted with Lys (K). In a particular embodiment, the Arg at position 562 is substituted with Asp (D), Glu (E), Asn (N), or Gln (Q). In a particular embodiment, the Arg at position 562 is substituted with Asn (N) or Gln (Q). In a particular embodiment, the Arg at position 562 is substituted with Gln (Q).

In certain embodiments, the Factor VIII variants comprise a mutation at position 519. In a particular embodiment, the Asp (D) at position 519 is not substituted with Glu (E). In a particular embodiment, the Asp at position 519 is substituted with Ala (A), Val (V), Ile (I), or Leu (L). In a particular embodiment, the Asp at position 519 is substituted with Val (V), Ile (I), or Leu (L). In a particular embodiment, the Asp at position 519 is substituted with Ala (A). In a particular embodiment, the Asp at position 519 is substituted with Val (V).

In certain embodiments, the Factor VIII variants comprise a mutation at position 665. In a particular embodiment, the Glu (E) at position 665 is not substituted with Asp (D). In a particular embodiment, the Glu at position 665 is substituted with Ala (A), Val (V), Ile (I), or Leu (L). In a particular embodiment, the Glu at position 665 is substituted with Val (V), Ile (I), or Leu (L). In a particular embodiment, the Glu at position 665 is substituted with Ala (A). In a particular embodiment, the Glu at position 665 is substituted with Val (V).

In certain embodiments, the Factor VIII variants further comprises a mutation at position 1984. In certain embodiments, the Factor VIII variant of the instant invention comprises a mutation at position 1984 instead of a mutation at 665. In a particular embodiment, the Glu (E) at position 1984 is not substituted with Asp (D). In a particular embodiment, the Glu at position 1984 is substituted with Ala (A), Val (V), Ile (I), or Leu (L). In a particular embodiment, the Glu at position 1984 is substituted with Val (V), Ile (I), or Leu (L). In a particular embodiment, the Glu at position 1984 is substituted with Ala (A). In a particular embodiment, the Glu at position 1984 is substituted with Val (V).

As stated hereinabove, the FVIII variant of the instant invention may be human. In a particular embodiment, the FVIII variant of the instant invention has at least 75%, 80%, 85%, 90%, 95%, 97%, 99%, or 100% homology (identity) with SEQ ID NO: 1 (or fragment or domain thereof or an activated FVIII fragment thereof), particularly at least 90%, 95%, 97%, 99%, or 100% homology (identity). In a particular embodiment, the FVIII variant comprises an amino acid sequence having at least 75%, 80%, 85%, 90%, 95%, 97%, 99%, or 100% homology (identity), particularly at least 90%, 95%, 97%, 99%, or 100% homology (identity), with amino acids 1-740 of SEQ ID NO: 1 (or fragment or domain thereof or an activated FVIII fragment thereof) and an amino acid sequence having at least 75%, 80%, 85%, 90%, 95%, 97%, 99%, or 100% homology (identity), particularly at least 90%, 95%, 97%, 99%, or 100% homology (identity) with amino acids 1649-2332 or 1690-2332 of SEQ ID NO: 1 (or fragment or domain thereof or an activated FVIII fragment thereof). The homology (identity) percentages above exclude the substitutions at position 336, 562, 519, and/or 665 (or 1984).

The FVIII variants of the instant invention may also be post-translationally modified. The FVIII variants may be post-translationally modified in a cell (particularly a human cell) or in vitro.

In certain embodiments, the FVIII variants of the invention have increased resistance to cleavage and/or inactivation (e.g., by APC and/or A2 dissociation) compared to wild-type FVIII.

In certain embodiments, the FVIII variants of the invention have increased specific activity compared to wild-type FVIII, FVIII-VV, and/or FVIII-QQ.

Nucleic acid molecules encoding the above FVIII variants (or fragments or domains thereof or activated fragments thereof) are also encompassed by the instant invention. Nucleic acid molecules encoding the variants may be prepared by any method known in the art. The nucleic acid molecules may be maintained in any convenient vector, particularly an expression vector.

Compositions comprising at least one FVIII variant and at least one carrier (e.g., pharmaceutically acceptable carrier) are also encompassed by the instant invention. In a particular embodiment, the FVIII is isolated and/or substantially pure within the composition. Compositions comprising at least one FVIII variant nucleic acid molecule and at least one carrier are also encompassed by the instant invention. Except insofar as any conventional carrier is incompatible with the variant to be administered, its use in the pharmaceutical composition is contemplated. In a particular embodiment, the carrier is a pharmaceutically acceptable carrier for intravenous administration.

Nucleic acid molecules encoding the variants of the invention may be prepared by using recombinant DNA technology methods. The availability of nucleotide sequence information enables preparation of isolated nucleic acid molecules of the invention by a variety of means. For example, nucleic acid sequences encoding a variant may be isolated from appropriate biological sources using standard protocols well known in the art.

Nucleic acids of the present invention may be maintained as RNA or DNA in any convenient cloning vector. In a particular embodiment, clones are maintained in a plasmid cloning/expression vector, which is propagated in a suitable host cell (e.g., E. coli). Alternatively, the nucleic acids may be maintained in a vector suitable for expression in mammalian cells. In cases where post-translational modification affects variant function, it is preferable to express the molecule in mammalian cells, particularly human cells.

FVIII variant encoding nucleic acid molecules of the invention include DNA, cDNA, genomic DNA, RNA, and fragments thereof which may be single- or double-stranded. Thus, this invention provides oligonucleotides (sense or antisense strands of DNA or RNA) having sequences capable of hybridizing with at least one sequence of a nucleic acid molecule of the present invention. Such oligonucleotides are useful as probes for detecting variant expression.

The FVIII variants of the present invention may be prepared in a variety of ways, according to known methods. The protein may be purified from appropriate sources (e.g., transformed bacterial or animal (e.g., mammalian or human) cultured cells or tissues which express FVIII variants), for example, by immunoaffinity purification or cation exchange chromatography purification. The availability of nucleic acid molecules encoding the variants enables production of the variants using in vitro expression methods known in the art. For example, a cDNA or gene may be cloned into an appropriate in vitro transcription vector followed by cell-free translation in a suitable cell-free translation system, such as wheat germ or rabbit reticulocyte lysates. In vitro transcription and translation systems are commercially available.

Alternatively, larger quantities of variant may be produced by expression in a suitable prokaryotic or eukaryotic expression system. For example, part or all of a DNA molecule encoding the FVIII variant may be inserted into a plasmid vector adapted for expression in a bacterial cell, such as E. coli, or a mammalian cell (particularly a human cell) such as CHO, BHK, or HeLa cells. Alternatively, tagged fusion proteins comprising the variant can be generated. Such variant-tagged fusion proteins are encoded by part or all of a DNA molecule, ligated in the correct codon reading frame to a nucleotide sequence encoding a portion or all of a desired polypeptide tag which is inserted into a plasmid vector adapted for expression in a bacterial cell, such as E. coli or a eukaryotic cell, such as, but not limited to, yeast and mammalian cells, particularly human cells. Vectors such as those described above comprise the regulatory elements necessary for expression of the DNA in the host cell positioned in such a manner as to permit expression of the DNA in the host cell. Such regulatory elements required for expression include, but are not limited to, promoter sequences, transcription initiation sequences, and enhancer sequences.

FVIII variant proteins, produced by gene expression in a recombinant prokaryotic or eukaryotic system (particularly human) may be purified according to methods known in the art. In a particular embodiment, a commercially available expression/secretion system can be used, whereby the recombinant protein is expressed and thereafter secreted from the host cell, to be easily purified from the surrounding medium. If expression/secretion vectors are not used, an alternative approach involves purifying the recombinant protein by affinity separation, such as by immunological interaction with antibodies that bind specifically to the recombinant protein or nickel columns for isolation of recombinant proteins tagged with 6-8 histidine residues at their N-terminus or C-terminus. Alternative tags may comprise, without limitation, the FLAG epitope, GST or the hemagglutinin epitope. Such methods are commonly used by skilled practitioners.

FVIII variant proteins, prepared by the aforementioned methods, may be analyzed according to standard procedures. For example, such proteins may be subjected to amino acid sequence analysis, according to known methods.

As discussed above, a convenient way of producing a polypeptide according to the present invention is to express nucleic acid encoding it, by use of the nucleic acid in an expression system. A variety of expression systems of utility for the methods of the present invention are well known to those of skill in the art.

Accordingly, the present invention also encompasses a method of making a polypeptide (as disclosed), the method including expression from nucleic acid encoding the polypeptide (generally nucleic acid). This may conveniently be achieved by culturing a host cell, containing such a vector, under appropriate conditions which cause or allow production of the polypeptide. Polypeptides may also be produced in in vitro systems, such as in reticulocyte lysates.

FVIII variant proteins and nucleic acids of the instant invention may be used, for example, as therapeutic and/or prophylactic agents which modulate the blood coagulation cascade. The FVIII variant proteins and nucleic acids of the instant invention may be administered in a therapeutically effective amount to modulate (e.g., increase) hemostasis and/or form a clot and/or stop or inhibit bleeding or aberrant bleeding. It is demonstrated herein that the FVIII variants possess superior properties and can provide effective hemostasis.

In a particular embodiment of the present invention, FVIII variants may be administered to a patient via infusion in a biologically compatible carrier, e.g., via injection or intravenous injection. The FVIII variants of the invention may optionally be encapsulated into liposomes or mixed with other phospholipids or micelles to increase stability of the molecule. FVIII variants may be administered alone or in combination with other agents known to modulate hemostasis (e.g., vFW, Factor IX, Factor IXa, etc.). An appropriate composition in which to deliver the FVIII variant may be determined by a medical practitioner upon consideration of a variety of physiological variables, including, but not limited to, the patient's condition and hemodynamic state. A variety of compositions well suited for different applications and routes of administration are well known in the art and are described hereinbelow.

The preparation containing the FVIII variants may contain a physiologically acceptable matrix and is formulated as a pharmaceutical preparation. The preparation can be formulated using substantially known methods, it can be mixed with a buffer containing salts, such as NaCl, CaCl₂, and amino acids, such as glycine and/or lysine, and in a pH range from 6 to 8. Until needed, the purified preparation containing the FVIII variant can be stored in the form of a finished solution or in lyophilized or deep-frozen form. In a particular embodiment, the preparation is stored in lyophilized form and is dissolved into a visually clear solution using an appropriate reconstitution solution. Alternatively, the preparation according to the present invention can also be made available as a liquid preparation or as a liquid that is deep-frozen. The preparation according to the present invention may be especially stable, i.e., it can be allowed to stand in dissolved form for a prolonged time prior to application.

The preparation according to the present invention can be made available as a pharmaceutical preparation with the FVIII variant in the form of a one-component preparation or in combination with other factors in the form of a multi-component preparation.

Prior to processing the purified protein into a pharmaceutical preparation, the purified protein may be subjected to the conventional quality controls and fashioned into a therapeutic form of presentation. In particular, during the recombinant manufacture, the purified preparation may be tested for the absence of cellular nucleic acids as well as nucleic acids that are derived from the expression vector.

Another feature of this invention relates to making available a preparation which contains a FVIII variant with a high stability and structural integrity and which, in particular, is free from inactive FVIII intermediates and/or proteolytic degradation products and and by formulating it into an appropriate preparation.

The pharmaceutical preparation may contain, as an example, dosages of between about 1-1000 μg/kg, about 10-500 μg/kg, about 10-250 μg/kg, or about 10-100 μg/kg. In a particular embodiment, the pharmaceutical protein preparation may comprise a dosage of between 30-100 IU/kg (e.g., as a single daily injection or up to 3 times or more/day). Patients may be treated immediately upon presentation at the clinic with a bleed or prior to the delivery of cut/wound causing a bleed. Alternatively, patients may receive a bolus infusion every one to three, eight, or twelve hours or, if sufficient improvement is observed, a once daily infusion of the FVIII variant described herein.

FVIII variant-encoding nucleic acids may be used for a variety of purposes in accordance with the present invention such as gene therapy and/or gene editing. In a particular embodiment of the invention, a nucleic acid delivery vehicle (e.g., a non-viral vector, lipid nanoparticles, an expression vector such as a viral vector, etc.) for modulating blood coagulation is provided wherein the expression vector comprises a nucleic acid sequence coding for a FVIII variant as described herein. Administration of the FVIII variant-encoding expression vectors to a patient results in the expression of the FVIII variant which serves to alter the coagulation cascade. In accordance with the present invention, a FVIII variant encoding nucleic acid sequence may encode a variant polypeptide as described herein whose expression increases hemostasis. In a particular embodiment, the nucleic acid sequence encodes a human FVIII variant.

Expression vectors comprising FVIII variant nucleic acid sequences may be administered alone, or in combination with other molecules useful for modulating hemostasis. According to the present invention, the expression vectors or combination of therapeutic agents may be administered to the patient alone or in a pharmaceutically acceptable or biologically compatible composition.

In a particular embodiment of the invention, the expression vector comprising nucleic acid sequences encoding the FVIII variant is a viral vector or non-viral vector. Viral vectors which may be used in the present invention include, but are not limited to, adenoviral vectors (with or without tissue specific promoters/enhancers), adeno-associated virus (AAV) vectors of any serotype (e.g., AAV-1 to AAV-12, particularly AAV-2, AAV-5, AAV-7, and AAV-8) and hybrid AAV vectors, lentivirus vectors and pseudo-typed lentivirus vectors (e.g., Ebola virus, vesicular stomatitis virus (VSV), and feline immunodeficiency virus (FIV)), herpes simplex virus vectors, vaccinia virus vectors, and retroviral vectors. Examples of non-viral means include, without limitation, gene delivery by lipid nanoparticles. In a particular embodiment, the vector is an adeno-associated virus (AAV) vector. In a particular embodiment, the vector is a lentiviral vector.

In a particular embodiment of the present invention, methods are provided for the administration of a vector (e.g., a non-viral or viral vector) comprising a nucleic acid sequence encoding a FVIII variant. Viral (e.g., AAV) vectors of utility in the methods of the present invention preferably include at least the essential parts of viral (e.g., AAV) vector DNA. As described herein, expression of a FVIII variant following administration of such a viral (e.g., AAV) vector serves to modulate hemostasis, particularly to enhance the procoagulation activity of the protease.

Recombinant viral (e.g., AAV) vectors have found broad utility for a variety of gene therapy applications. Their utility for such applications is due largely to the high efficiency of in vivo gene transfer achieved in a variety of organ contexts.

AAV particles may be used to advantage as vehicles for adequate gene delivery. Such virions possess a number of desirable features for such applications, including: structural features related to being a double stranded DNA nonenveloped virus and biological features such as a tropism for the human respiratory system and gastrointestinal tract. Moreover, AAV are known to infect a wide variety of cell types in vivo and in vitro by receptor-mediated endocytosis. Attesting to the overall safety of AAV vectors, infection with AAV leads to a minimal disease state in humans comprising mild flu-like symptoms.

Viral (e.g., AAV) genomes are well suited for use as gene therapy vehicles because they can accommodate the insertion of foreign DNA following the removal of viral genes essential for replication and/or nonessential regions. Such substitutions render the viral vector impaired with regard to replicative functions and infectivity. Many viruses (e.g., AAV) have been used as vectors for gene therapy and for expression of heterologous genes.

It is desirable to introduce a vector that can provide, for example, multiple copies of a desired gene and hence greater amounts of the product of that gene. Improved viral (e.g., AAV) vectors and methods for producing these vectors have been described (e.g., Penn Vector Core; addgene; etc.).

For some applications, an expression construct may further comprise regulatory elements which serve to drive expression in a particular cell or tissue type and/or constitutively. Such regulatory elements are known to those of skill in the art. The incorporation of tissue specific regulatory elements in the expression constructs of the present invention provides for at least partial tissue tropism for the expression of the variant or functional fragments thereof. In certain embodiments, a constitutive promoter (e.g., cytomegalovirus (CMV) promoter) may be used. Hematopoietic or liver specific promoters may also be used.

AAV for recombinant gene expression have been produced in human cells (e.g., the human embryonic kidney cell line 293). Briefly, AAV vectors are typically engineered from wild-type AAV, a single-stranded DNA virus that is non-pathogenic. The parent virus is non-pathogenic, the vectors have a broad host range, and they can infect both dividing and non-dividing cells. The vector is typically engineered from the virus by deleting the rep and cap genes and replacing these with the transgene of interest under the control of a specific promoter. For recombinant AAV preparation, the upper size limit of the sequence that can be inserted between the two ITRs is about 4.7 kb. Plasmids expressing a FVIII variant under the control of a promoter (e.g., the CMV promoter/enhancer) and a second plasmid supplying adenovirus helper functions along with a third plasmid containing the rep and cap genes (e.g., AAV-2 rep and cap genes) may be used to produce AAV vectors (e.g., AAV-2 vectors). Other AAV serotype cap genes (e.g., AAV-1, AAV-6, or AAV-8 cap genes) may be expressed with other serotype rep genes and ITRs (e.g., AAV-2 rep gene and ITRs) to produce different vectors (e.g., Gao et al. (2002) Proc. Natl. Acad. Sci. USA 99:11854-11859; Xiao et al., (1999) J. Virol. 73:3994-4003; Arruda et al., (2004) Blood 103:85-92). AAV vectors may be purified by repeated CsCl density gradient centrifugation and the titer of purified vectors determined by quantitative dot-blot hybridization. In a particular embodiment, vectors may be prepared by the Vector Core at The Children's Hospital of Philadelphia.

Also included in the present invention is a method for modulating hemostasis comprising providing cells of an individual with a nucleic acid delivery vehicle encoding a FVIII variant and allowing the cells to grow under conditions wherein the FVIII variant is expressed.

From the foregoing discussion, it can be seen that FVIII variants and FVIII variant encoding nucleic acid molecules (e.g., FVIII variant expressing nucleic acid vectors) may be used in the treatment of disorders associated with aberrant blood coagulation.

FVIII variant encoding nucleic acid molecules (e.g., expression vectors) of the present invention may be incorporated into pharmaceutical compositions that may be delivered to a subject, so as to allow production of a biologically active protein (e.g., a FVIII variant) or by inducing expression of the FVIII variant in vivo by gene- and or cell-based therapies or by ex vivo modification/transduction of the patient's or donor's cells. The FVIII variant encoding nucleic acid molecules may be used for gene addition or gene editing to express the FVIII variants of the instant invention (e.g., FVIII-QQVV). In certain embodiments, gene editing comprises altering the FVIII gene in a subject to insert or substitute the FVIII variants of the instant invention (e.g., FVIII-QQVV). Gene editing tools are known in the art and include, without limitation, zinc finger nucleases, TALEN (Transcription Activator-Like Effector Nucleases), and CRISPR (Clustered Regulatory Interspaced Short Palindromic Repeats)/Cas9 gene editing. In certain embodiments, the FVIII gene is edited using CRISPR/Cas9 technology. In certain embodiments, CRISPR mediated gene editing may utilize non-homologous end-joining (NHEJ) or homologous recombination to affect the gene editing.

In a particular embodiment of the present invention, pharmaceutical compositions comprising sufficient genetic material to enable a recipient to produce a therapeutically effective amount of a FVIII variant can influence hemostasis in the subject. Alternatively, as discussed above, an effective amount of the FVIII variant may be directly infused into a patient in need thereof. The compositions may be administered alone or in combination with at least one other agent, such as a stabilizing compound, which may be administered in any sterile, biocompatible pharmaceutical carrier, including, but not limited to, saline, buffered saline, dextrose, and water. The compositions may be administered to a patient alone, or in combination with other agents (e.g., co-factors) which influence hemostasis.

In particular embodiments, the compositions (e.g., pharmaceutical compositions) of the instant invention also contain a pharmaceutically acceptable carrier. Such carriers include any pharmaceutical agent that does not itself induce an immune response harmful to the individual receiving the composition, and which may be administered without undue toxicity. Pharmaceutically acceptable carriers include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington's Pharmaceutical Sciences.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding, free base forms. In other cases, the preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

After pharmaceutical compositions have been prepared, they may be placed in an appropriate container and labeled for treatment. For administration of FVIII variants or FVIII variant encoding nucleic acids (e.g., vectors), such labeling could include amount, frequency, and method of administration.

Pharmaceutical compositions suitable for use in the invention include compositions wherein the active ingredients are contained in an effective amount to achieve the intended therapeutic purpose. Determining a therapeutically effective dose is well within the capability of a skilled medical practitioner using the techniques and guidance provided in the present invention. Therapeutic doses will depend on, among other factors, the age and general condition of the subject, the severity of the aberrant blood coagulation phenotype, and the strength of the control sequences regulating the expression levels of the variant polypeptide. Thus, a therapeutically effective amount in humans will fall in a relatively broad range that may be determined by a medical practitioner based on the response of an individual patient to vector-based variant treatment.

The FVIII variants, alone or in combination with other agents, may be directly infused into a patient in an appropriate biological/pharmaceutical carrier as described hereinabove. Expression vectors of the present invention comprising nucleic acid sequences encoding variant or functional fragments thereof, may be administered to a patient by a variety of means (see below) to achieve and maintain a prophylactically and/or therapeutically effective level of the variant polypeptide. One of skill in the art could readily determine specific protocols for using the variant encoding expression vectors of the present invention for the therapeutic treatment of a particular patient.

FVIII variants and/or FVIII variant encoding nucleic acids (e.g., AAV vectors) of the present invention may be administered to a patient by any means known. Direct delivery of the pharmaceutical compositions in vivo may generally be accomplished via injection using a conventional syringe, although other delivery methods such as convection-enhanced delivery are envisioned. In this regard, the compositions may be delivered subcutaneously, epidermally, intradermally, intrathecally, intraorbitally, intramucosally, intraperitoneally, intravenously, intraarterially, orally, intrahepatically or intramuscularly. Other modes of administration include oral and pulmonary administration, suppositories, and transdermal applications. In certain embodiments, the FVIII is administered by injection (e.g., to the bloodstream). In certain embodiments, the FVIII variant encoding nucleic acids (e.g., AAV vectors) is administered by injection (e.g., to the bloodstream or liver). A clinician specializing in the treatment of patients with blood coagulation disorders may determine the optimal route for administration of the vectors (e.g., AAV vectors) comprising variant nucleic acid sequences based on a number of criteria, including, but not limited to: the condition of the patient and the purpose of the treatment (e.g., reduced blood coagulation).

The present invention also encompasses vectors (e.g., viral vectors or AAV vectors) comprising a nucleic acid sequence encoding a FVIII variant. Also provided are lentiviruses or pseudo-typed lentivirus vectors comprising a nucleic acid sequence encoding a FVIII variant. Also encompassed are naked plasmid or expression vectors comprising a nucleic acid sequence encoding a FVIII variant.

### Definitions

The following definitions are provided to facilitate an understanding of the present invention.

The singular forms “a,” “an,” and “the” include plural referents unless the context clearly dictates otherwise.

The phrase “hemostasis related disorder” refers to bleeding disorders such as, without limitation, hemophilia A, hemophilia B, hemophilia A and B patients, hemophilia with inhibitory antibodies, deficiencies in at least one coagulation factor (e.g., Factors VII, VIII, IX, X, XI, V, XII, II, and/or von Willebrand factor, particularly Factor VIII), combined FV/FVIII deficiency, vitamin K epoxide reductase C1 deficiency, gamma-carboxylase deficiency, bleeding associated with trauma or injury, thrombosis, thrombocytopenia, stroke, coagulopathy (hypocoagulability), disseminated intravascular coagulation (DIC), over-anticoagulation associated with heparin, low molecular weight heparin, pentasaccharide, warfarin, or small molecule antithrombotics (e.g., FXa inhibitors); and platelet disorders such as, Bernard Soulier syndrome, Glanzman thromblastemia, and storage pool deficiency. In a particular embodiment, the term “hemostasis related disorder” refers to bleeding disorders characterized by excessive and/or uncontrolled bleeding (e.g., a disorder which can be treated with a procoagulant). In a particular embodiment, the hemostasis related disorder is hemophilia. In a particular embodiment, the hemostasis related disorder is hemophilia A.

With reference to nucleic acids of the invention, the term “isolated nucleic acid” is sometimes used. This term, when applied to DNA, refers to a DNA molecule that is separated from sequences with which it is immediately contiguous (in the 5′ and 3′ directions) in the naturally occurring genome of the organism from which it originates. For example, the “isolated nucleic acid” may comprise a DNA or cDNA molecule inserted into a vector, such as a plasmid or virus vector, or integrated into the DNA of a prokaryote or eukaryote. With respect to RNA molecules of the invention, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form.

With respect to protein, the term “isolated protein” is sometimes used herein. This term may refer to a protein produced by expression of an isolated nucleic acid molecule of the invention. Alternatively, this term may refer to a protein which has been sufficiently separated from other proteins with which it would naturally be associated (e.g., so as to exist in “substantially pure” form). “Isolated” is not meant to exclude artificial or synthetic mixtures with other compounds or materials, or the presence of impurities that do not interfere with the fundamental activity, and that may be present, for example, due to incomplete purification, or the addition of stabilizers.

The term “vector” refers to a carrier nucleic acid molecule (e.g., RNA or DNA) into which a nucleic acid sequence can be inserted for introduction into a host cell where it will be replicated. An “expression vector” is a specialized vector that contains a gene or nucleic acid sequence with the necessary regulatory regions (e.g., promoter) needed for expression in a host cell.

The term “operably linked” means that the regulatory sequences necessary for expression of a coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule wherein a hybrid nucleic acid molecule is generated.

The term “substantially pure” refers to a preparation comprising at least 50-60% by weight the compound of interest (e.g., nucleic acid, oligonucleotide, protein, etc.), particularly at least 75% by weight, or at least 90-99% or more by weight of the compound of interest. Purity may be measured by methods appropriate for the compound of interest (e.g. chromatographic methods, agarose or polyacrylamide gel electrophoresis, HPLC analysis, and the like).

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., Tris HCl, acetate, phosphate), antimicrobial, bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin (Mack Publishing Co., Easton, PA); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

As used herein, the term “subject” refers to an animal, particularly a mammal, particularly a human.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “prevent” refers to the prophylactic treatment of a subject who is at risk of developing a condition (e.g., aberrant bleeding) resulting in a decrease in the probability that the subject will develop the condition.

A “therapeutically effective amount” of a compound or a pharmaceutical composition refers to an amount effective to prevent, inhibit, treat, and/or lessen the symptoms of a particular disorder or disease.

As used herein, “gene editing” refers to genetic engineering in which the nucleotide sequence of a target polynucleotide is changed through introduction of deletions, insertions, or base substitutions to the polynucleotide sequence.

The following example is provided to illustrate various embodiments of the present invention. The example is illustrative and is not intended to limit the invention in any way.

### Example

The cofactor function of plasma protein factor VIII (FVIII) is crucial to the propagation and amplification of the coagulation cascade, and deficiency or dysfunction of the protein results in hemophilia A (HA), the most common of severe bleeding disorders (Mann, et al. (1998) Annual Review of Biochemistry, 57:915-956; van Dieijen, et al. (1981) J. Biol. Chem., 256:3433-3442). FVIII circulates in blood tightly bound to its carrier protein, von Willebrand factor (VWF), and upon proteolytic processing by thrombin, is liberated from VWF to produce its active cofactor form (FVIIIa) (Eaton, et al. (1986) Biochemistry 25:505-512; Vehar, et al. (1984) Nature 312:337-342; Fay, et al. (1992) J. Biol. Chem., 267:13246-13250; Lollar, et al. (1988) J. Biol. Chem., 263:10451-10455). FVIIIa exists as a heterotrimer comprised of an A2-domain weakly associated with the metal ion-stabilized A1/A3-C1-C2 heterodimer and associates with factor IXa (FIXa) on negatively charged phospholipid surfaces to form the intrinsic factor Xase (intrinsic Xase) enzyme complex, which efficiently converts zymogen factor X (FX) into serine protease factor Xa (FXa) (Eaton, et al. (1986) Biochemistry 25:505-512; Hill-Eubanks, et al. (1990) J. Biol. Chem., 265:17854-17858; Kolkman, et al. (1999) J. Biol. Chem., 274:29087-29093; Kolkman, et al. (1999) Biochem. J., 339 (Pt 2): 217-221; Kolkman, et al. (2000) Biochemistry 39:7398-7405; Lenting, et al. (1994) J. Biol. Chem., 269:7150-7155; Mann, et al. (1990) Blood 76:1-16; Venkateswarlu, D. (2014) Biochem. Biophys. Res. Commun., 452:408-414; Fay, et al. (1998) J. Biol. Chem., 273:19049-19054). Acting as a cofactor, FVIIIa increases the function of FIXa by 3- to 6-orders of magnitude, and thus its inactivation is thought to be an important regulator of intrinsic Xase function (van Dieijen, et al. (1981) J. Biol. Chem., 256:3433-3442; Mertens, et al. (1984) Biochem. J., 223:599-605; Fay, et al. (1996) J. Biol. Chem., 271:6027-6032).

Two modes of FVIIIa inactivation are spontaneous A2-domain dissociation and proteolytic cleavage by activated protein C (APC) at Arg336 and Arg562 within the A1 and A2 domains, respectively (Fay, et al. (1991) J. Biol. Chem., 266:20139-20145; Hultin, et al. (1981) Blood 57:476-482; Lollar, et al. (1984) Blood 63:1303-1308; Lollar, et al. (1990) J. Biol. Chem., 265:1688-1692; Lollar, et al. (1991) J. Biol. Chem., 266:12481-12486; Plautz, et al. (2018) Arterioscler. Thromb. Vasc. Biol., 38:816-828; Walker, et al. (1987) Arch. Biochem. Biophys., 252:322-328; Lollar, et al. (1994) J. Clin. Invest., 93:2497-2504). The A2 subunit of FVIIIa is weakly associated (Kd˜260 nM) with the heterodimer mainly through electrostatic interactions, and under physiologic conditions it readily dissociates within minutes resulting in a loss of FVIIIa activity (Fay, et al. (1992) J. Biol. Chem., 267:13246-13250; Lollar, et al. (1991) J. Biol. Chem., 266:12481-12486; Lollar, et al. (1994) J. Clin. Invest., 93:2497-2504; Fay, et al. (1991) J. Biol. Chem., 266:8957-8962; Lollar, et al. (1992) J. Biol. Chem., 267:23652-23657; Parker, et al. (2006) J. Biol. Chem., 281:13922-13930; Leong, et al. (2015) Blood 125:392-398; Wakabayashi, et al. (2008) Blood 112:2761-2769). The significance of nonproteolytic inactivation of FVIIIa is highlighted in individuals with mutations at A2 domain interaction sites, causing decreased A2 domain affinity for the heterodimer, having mild HA phenotypes (Duncan, et al. (1994) Brit. J. Haematology 87:846-848; Hakeos, et al. (2002) Thrombosis Haemostasis 88:781-787; McGinniss, et al. (1993) Genomics 15:392-398; Pipe, et al. (1999) Blood 93:176-183; Pipe, et al. (2001) Blood 97:685-691; Rudzki, et al. (1996) Brit J. Haematology 94:400-406). Contrastingly, APC-mediated inactivation of FVIIIa occurs over hours and clinical phenotypes associated with altered APC cleavage of FVIII/FVIIIa are lacking (Fay, et al. (1991) J. Biol. Chem., 266:20139-20145; Bezemer, et al. (2008) JAMA 299:1306-1314; Lu, et al. (1996) Blood 87:4708-4717; O'Brien, et al. (2000) Blood 95:1714-1720). The faster rate of FVIIIa inactivation attributed to A2-domain dissociation compared to that of APC cleavage has implicated the former as the predominant mechanism of FVIIIa inactivation; however, the impact of physiologic ligands on these mechanisms is incompletely understood (Hultin, et al. (1981) Blood 57:476-482; Lollar, et al. (1984) Blood 63:1303-1308; Lollar, et al. (1990) J. Biol. Chem., 265:1688-1692; Lollar, et al. (1991) J. Biol. Chem., 266:12481-12486; Fay, et al. (1991) J. Biol. Chem., 266:8957-8962; Leong, et al. (2015) Blood 125:392-398; Wakabayashi, et al. (2008) Blood 112:2761-2769; Lu, et al. (1996) Blood 87:4708-4717; O'Brien, et al. (2000) Blood 95:1714-1720).

FVIIIa is considerably more stable in the presence of its enzyme, FIXa (Lenting, et al. (1994) J. Biol. Chem., 269:7150-7155; Lollar, et al. (1984) Blood 63:1303-1308; Fay, P. J. (1999) Thromb. Haemost., 82:193-200; Fay, et al. (1994) J. Biol. Chem., 269:20522-20527; Lenting, et al. (1996) J. Biol. Chem., 271:1935-1940). Interactions between FVIIIa and FIXa on phospholipid membranes are mediated by a high (Kd˜ 14 nM) and low (Kd˜300 nM) affinity FIXa binding site located on the A3 domain, residues 1811-1818, and A2 domain, residues 558-565, respectively (Lenting, et al. (1994) J. Biol. Chem., 269:7150-7155; Fay, et al. (1994) J. Biol. Chem., 269:20522-20527; Lenting, et al. (1996) J. Biol. Chem., 271:1935-1940). Binding of FVIIIa to FIXa increases the half-life of FVIIIa approximately 10-fold, suggesting FIXa serves as a bridge linking the A2 and A1/A3-C1-C2 subunits to reduce the rate of A2-domain dissociation from the FVIIIa heterodimer (Lollar, et al. (1984) Blood 63:1303-1308; Fay, P. J. (1999) Thromb. Haemost., 82:193-200). Binding of the cofactor/enzyme complex to its substrate, FX, has also been shown to increase the stability of FVIIIa cofactor function (O'Brien, et al. (2000) Blood 95:1714-1720; Lapan, et al. (1997) J. Biol. Chem., 272:2082-2088; Regan, et al. (1996) J. Biol. Chem., 271:3982-3987). Interestingly, the identification of a FX interactive site on FVIIIa within the A1 domain, residues 337-372, adjacent to APC cleavage site Arg336 suggests that the stabilizing effects of FX are APC cleavage related; although, remarks on the relative contributions of modes of FVIIIa inactivation have lacked proper controls (O'Brien, et al. (2000) Blood 95:1714-1720; Lapan, et al. (1997) J. Biol. Chem., 272:2082-2088; Regan, et al. (1996) J. Biol. Chem., 271:3982-3987).

To study the role of intrinsic Xase ligands, FIXa and FX, in the regulation of FVIIIa, a series was generated of recombinant FVIII variants resistant to APC cleavage (FVIII R336Q, R562Q; FVIII-QQ), A2-domain dissociation (FVIII D519V, E665V; FVIII-VV), or both (FVIII-QQ/VV). Herein, the novel control FVIII species are utilized to probe each mechanism of FVIIIa inactivation independently (Wakabayashi, et al. (2009) J. Thrombosis Haemostasis 7:438-444; Wilhelm, et al. (2021) Blood 137:2532-2543). It is shown herein that FVIIIa-FIXa interactions confer increased stability on FVIIIa that are further enhanced by the presence of FX under physiologically relevant conditions. Furthermore, it is demonstrated that the addition of FX in the intrinsic Xase enzyme complex abrogates APC-mediated inactivation of FVIIIa. Notably, a 4- to 5-fold improvement in the hemostatic effect of FVIII species resistant to APC is seen in vivo. Consequently, these findings indicate that FVIIIa exists in intermediate states of ligand saturation and provide insight on a beneficial mechanism that can be targeted for therapeutic purpose.

### Materials and Methods

### Reagents

The inhibitors benzamidine and 4-amidinophenylmethanesulfonyl fluoride hydrochloride (APMSF) were obtained from Sigma Aldrich (St. Louis, MO). Hepes, Tris buffers, bovine serum albumin (BSA), Tween 80 and all other reagents, not indicated otherwise, were also purchased from Sigma. Cell culture reagents were from Invitrogen (Waltham, MA) except for insulin-transferrin-sodium selenite which was purchased from Roche (Basel, Switzerland). Synthetic phospholipid vesicles (PCPS) were prepared from 75% hen egg L-α-phosphatidylcholine (PC) and 25% porcine brain L-α-phosphatidylserine (PS) (Avanti Polar Lipids; Alabaster, AL) and quantified as described (Pittman, et al. (1993) Blood 81 (11): 2925-2935). Triniclot reagent (Tcoag) was used to measure automated activated partial thromboplastin time (aPTT). The peptidyl substrate, Spectrozyme® Xa (Sekisui Diagnostics; Burlington, MA), was prepared in water and concentration was verified using E342=8279 M⁻¹cm⁻¹ (Lottenberg, et al. (1983) Biochim. Biophys. Acta., 742 (3): 558-564). The fluorogenic substrate, 0.5 mM Z-Gly-Gly-Arg-AMC, was purchased from Bachem Bioscience Inc. (Bubendorf, Switzerland), was reconstituted in 15 mM CaCl₂) and the concentration was determined using E326=17,200 M⁻¹cm⁻¹ (Bunce, et al. (2011) Blood 117 (1): 290-298). Pooled-platelet-poor normal human plasma and FVIII deficient plasma were purchased from George King Biomedical (Overland Park, KS). Unless noted otherwise, all assays were done at 25° C. in assay buffer (20 mM HEPES [4-(2-hydroxyethyl)-1-piperazineethanesulfonic acid], 150 mM NaCl, 5 mM CaCl₂, 0.1% polyethylene glycol-8000, pH 7.4) and all listed reagent or protein concentrations are final concentrations for experimental conditions.

### Proteins

Plasma-derived FX, FXa, FX-R15Q (enzymatically inert), and thrombin were purified and prepared as described (Baugh, et al. (1996) J. Biol. Chem., 271:16126-16134; Buddai, et al. (2002) J. Biol. Chem., 277 (29): 26689-26698; Basavaraj, et al. (2020) J. Biol. Chem., 295:15198-15207). Factor IXa, active site blocked FIXa (FIXa-DEGR), FXIa, PS, and APC were purchased from Haemtech (Essex Junction, VT) or Enzyme Research (South Bend, IN). Hirudin was purchased from Calbiochem (San Diego, CA). Protein concentrations were determined immediately before each experiment using the following molecular weights (Mr) and extinction coefficient (E)^{0.1%}. thrombin (37,500 and 1.94), FIXa and FIXa-DEGR (45,000 and 1.40), FX and FX-R15Q (58,900 and 1.16), FXa (46,000 and 1.16), thrombin (37,500 and 1.94), FXIa (160,000 and 1.34), APC (56,200 and 1.45), and PS (69,000 and 0.95), respectively (Basavaraj, et al. (2020) J. Biol. Chem., 295:15198-15207; Lundblad, et al. (1976) Thrombin 1976:156-176; Di Scipio, et al. (1977) Biochemistry 16 (4): 698-706; Fujikawa, et al. (1974) Biochemistry 13 (22): 4508-4516).

### Generation of Recombinant FVIII Proteins

Baby hamster kidney (BHK) cell lines stably expressing wild-type B-domain deleted FVIII (FVIII-WT) were developed and recombinant protein was purified by established procedure (Pittman, et al. (1993) Blood 81 (11): 2925-2935; Sabatino, et al. (2009) Blood 114 (20): 4562-4565). Site-directed mutagenesis of FVIII-WT cDNA (Genescript; Piscataway, NJ) was employed to introduce Arg to Gln mutations at FVIII APC cleavage sites, Arg336 and Arg562, and employed to introduce D519V and E665V substitutions; or both (FIG. 1). Factor VIII proteins were purified (˜3 mg each) from 24 liters of conditioned media using ion-exchange chromatography (Sabatino, et al. (2009) Blood 114 (20): 4562-4565). Recombinant FVIII concentrations were determined by absorbance at 280 nm based on an E0.1% of 1.60 and molecular weight (Mr)=165,000 (Curtis, et al. (1994) J. Bio. Chem., 269 (8): 6246-6251).

### Plasma Assays

FVIII specific activity was determined by an aPTT-based-1-stage clotting assay and by chromogenic assay (Siner, et al. (2016) JCI Insight., 1 (16): e89371). Residual cofactor activity and thrombin generation in platelet-poor FVIII-deficient plasma was determined as described with modifications (Bunce, et al. (2011) Blood 117 (1): 290-298). Factor VIII-deficient plasma was reconstituted with 1 nM FVIII or 0.2 nM FVIIIa with 4 μM PCPS. To generate FVIIIa, FVIII (1.5 nM) was incubated with thrombin (30 nM) for 30 seconds and quenched with hirudin (60 nM). In FVIII reconstituted plasma, thrombin generation was initiated using 1 pM or 30 pM FXIa in human and murine plasma, respectively. In FVIIIa reconstituted plasma, thrombin generation was initiated with 10 pM FXIa and 400 pM FXIa in human and mouse plasma, respectively. The concentration of FVIIIa and FXIa in these assays were chosen to generate similar peak thrombin and lag times relative to experiments with FVIII in analogous HA plasma. Peak thrombin accumulation was observed with 0.5 mM Z-Gly-Gly-Arg-AMC (Bachem Bioscience Inc.) in 7.5 mM CaCl₂). Fluorescence was measured over 90 minutes at 37° C. or 33° C. for human and mouse plasma, respectively, by a Spectromax® M2 (Molecular Devices; San Jose, CA) with 360 nm excitation and 460 nm emission wavelengths. Raw fluorescence values were compared to a thrombin calibration curve using a thrombin calibrator (Technothrombin® thrombin generation assay calibrator set) to convert data to nM thrombin and thrombin generation curves (nM/time) and analyzed to determine peak thrombin generation and lag time. APC was used because human soluble thrombomodulin (sTm) does not cross-react with mouse APC.

### SDS PAGE and Immunoblotting Analysis of FVIII Proteins in the Presence of APC

FVIIIa was generated by incubating FVIII (1.5 μM) with thrombin (10 nM) for 20 minutes and then quenched with hirudin (20 nM). Proteolytic cleavage by APC was evaluated (Wilhelm, et al. (2021) Blood 137:2532-2543). Briefly, FVIII (10 nM) was incubated with APC (6 nM), hirudin (6 nM), and PCPS (20 μM) for 30 minutes. Proteins and protein fragments were subjected to gel electrophoresis using 4% to 12% gradient NuPage gels (Invitrogen) under reducing conditions using Mops. Proteins were then transferred onto nitrocellulose membranes using a dry iBlot2® system (Invitrogen) followed by blocking with western blocking reagent (roche). Membranes were probed with a primary antibody that recognizes the FVIII A2-domain (GMA-012; Green Mountain Antibodies) and IRDyLight™ 800 secondary antibody (Rockland) (Fay, et al. (1991) J. Biol. Chem., 266:8957-8962).

### Factor VIII Enzyme Kinetic Studies

Kinetic analysis of FXa generation was performed by an intrinsic Xase assay, as described with modifications (Wilhelm, et al. (2021) Blood 137:2532-2543; Lollar, et al. (1989) Biochemistry 28 (2): 666-674). Activated FVIII (FVIIIa) was generated by incubating 25 nM FVIII with 100 nM thrombin for 30 seconds and thereafter quenched with hirudin (150 nM). Thereafter, FVIIIa (0.25 nM) was immediately combined with PCPS (20 μM) and FIXa (20 nM) with escalating concentrations of FX (0-500 nM) to determine Kₘ, and escalating concentrations of FIXa (0-20 nM) with FX (200 nM) to determine K_{d}. At various time intervals (0.25-2 minutes), aliquots of the reaction mixture were quenched in 20 mM HEPES, 150 mM NaCl, 25 mM EDTA, 0.1% polyethylene glycol-8000, pH 7.4. The amount of FXa in each quenched sample was assessed using Spectrozyme® Xa by measuring absorbance at 405 nm in SpectraMax® 190 Microplate reader (Molecular Devices) and comparing the results to a prepared FXa standard curve.

### Kinetics of FVIII A2-Domain Stability

Evaluation of FVIIIa A2-domain dissociation was performed as described with the following modifications. FVIII (5 nM) were first activated with thrombin (100 nM), then aliquots were taken at indicated time points and assayed immediately for residual FVIIIa function in the intrinsic Xase assay (Lollar, et al. (1989) Biochemistry 28 (2): 666-674).

### Kinetics of FVIII Cleavage by APC

Residual FVIII activity in the presence of APC was determined as described with the following modifications. FVIII (10 nM) were incubated with and without APC (6 nM) in the presence of PCPS (20 μM) and hirudin (6 nM) for 0 to 60 minutes prior to thrombin activation.

Kinetics of Fractional Saturation of FVIIIa by FIXa with and without FX on FVIIIa Stability

To evaluate the effect of physiologic ligands on the stability of FVIIIa, residual FVIII activity following the fractional saturation of FVIIIa with FIXa in the presence and absence of FX was determined as described with the following modifications. At indicated time points over 30 minutes, FVIII (5 nM) was activated with thrombin (100 nM) in the presence of PCPS (80 μM) and enzymatically inert FIXa (FIXa-DEGR; 0-10 nM). Approximate percentages of FVIIIa bound to FIXa-DEGR were determined using a Kd for FVIIIa-FIXa binding of 1 nM (previously described and confirmed by experiments not shown). Following the addition of hirudin (6 nM), enzymatically inert FX (FX-R15Q; 20 nM), and either a combination of APC (6 nM), protein S (50 nM), and hirudin (6 nM) or buffer was added to the reaction and incubated for 0-30 minutes prior to addition into the intrinsic Xase assay.

### Data Analysis

Analyses were performed in Graphpad Prism 9 software. Steady-state kinetic parameters, Kₘ, Kd, and Vₘₐₓ, for FX activation by the intrinsic FXase were calculated by non-weighted nonlinear least-squares fits to the Michaelis-Menten equation. Results are expressed as ±standard error of the mean.

### Results

FVIII has a plasma concentration of 1 nM and is activated to FVIIIa. FVIIIa cofactor functions within the intrinsic Xase enzyme complex and increases FIXa enzymatic function 103-106 fold, converting FX to FXa for FIXa. There are two described mechanisms of FVIIIa inactivation: 1) A2 domain dissociation and 2) cleavage by Activated Protein C (APC) and possibly protein S (PS). FVIIIa heterotrimer is weakly associated with A2 by weak electrostatic interactions and A2 dissociates with a Ka of 300 nM.

FVIIIa can be stabilized and A2 dissociation reduced by elimination of charged residues at the A1-A2 and/or A2-A3 interfaces (Leong et al. (2015) Blood 125:392-398; Wakabayashi, et al. (2009) J. Thromb. Haemost., 7 (3): 438-444; Wakabayashi, et al. (2008) Blood 112 (7): 2761-2769; Wakabayashi, et al. (2008) J. Biol. Chem., 283:11645-11651). These stabilized FVIII molecules retained their activities longer than wild-type FVIIIa. One noncovalent stabilization variant, D519V/E665V, which was presented in B domain-deleted FVIII (BDD-FVIII), exhibited a two-fold increase in activity as determined by the 2-stage chromogenic assay relative to BDD-FVIII and a two-fold increase in clotting potency in a mouse tail clip assay (Leong et al. (2015) Blood 125:392-398).

Various assays were performed to determine if any synergy was associated with the combination of mutations which impart resistance to APC cleavage (e.g., QQ mutations (R336Q/R562Q)) with mutations which reduce A2 domain dissociation or improve A2 domain affinity (e.g., VV mutations (D519V/E665V)).

FIGS. 1B and 1C provide schematic representations of FVIII proteins used herein. All variants were generated from site-directed mutagenesis of human B-domain deleted FVIII-WT complementary DNA (Genescript) to introduce Arg to Gln mutations at APC cleavage sites Arg336 and Arg562 (FVIII-QQ), Asp/Glu to Val mutations at A2 domain associated residues Asp519 and Glu665 (FVIII-VV), or both (FVIII-QQVV).

FVIII proteins were purified from conditioned media in their single chain (Mr=165,000) and heterodimeric forms (heavy chain, Mr=90,000; light chain, Mr=80,000) and migrated at the expected positions on a reduced SDS Page gel (FIG. 1D). Thrombin activation of FVIII proteins yielded fragments consistent with FVIIIa, representing cleavage at Arg1689 (A3-C1-C2, Mr=70,000) and Arg740/Arg372 (A1, Mr=50,000; A2, Mr=43,000; FIG. 1D).

FVIII variant resistance to proteolytic cleavage at APC cleavage sites Arg336 and Arg562 was confirmed by incubating FVIII proteins with APC for 30 minutes and evaluating reaction products by western blot analysis. As expected, APC cleavage of FVIII-WT and FVIII-VV yielded fragments consistent with both APC cleavage sites, while no similar cleavage fragments were detected for APC resistant species, FVIII-QQ and FVIII-QQ/VV (FIG. 1E).

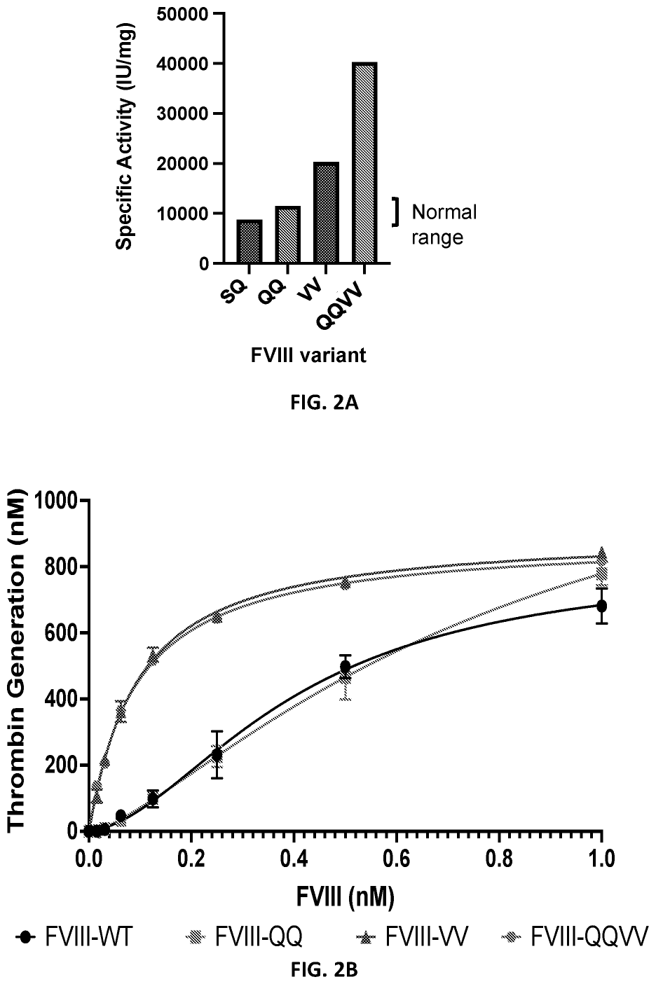

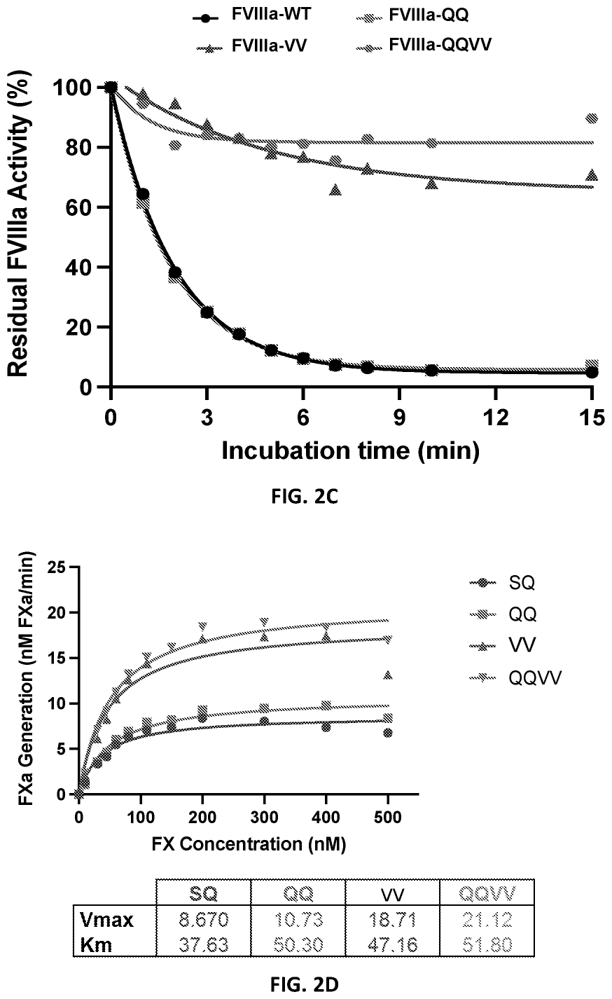

A one-stage assay was performed on various FVIII variants to determine their specific activity. Specifically, the specific activities of FVIII-SQ (also referred to as WT), FVIII-QQ, FVIII-VV, and FVIII-QQVV were determined. As seen in FIG. 2A, the specific activity of FVIII-SQ and FVIII-QQ were similar and consistent with FVIII-WT (9000+700 IU/mg) and commercially available B-domainless FVIII products (www.fda.gov/media/70399/download2014). Notably, the specific activity of FVIII-VV was about 2-fold greater than the specific activity of FVIII-SQ and FVIII-QQ. Surprisingly, the specific activity of FVIII-QQVV was about 2-fold greater than the specific activity of FVIII-VV even in the absence of APC and about 4-fold greater than the specific activity of FVIII-SQ. Indeed, in the absence of APC, the QQ mutations (which impart resistance to APC cleavage) would not have been expected to increase the specific activity of FVIII-VV.

FVIII proteins were biochemically characterized in a purified system demonstrating the same Km (˜50 nM) and less than 2-fold Vmax variability (˜8-20 nM FXa/min) for FX activation by intrinsic Xase assay (Table 1). These values are consistent with published data (Lollar, et al. (1994) J. Clin. Invest., 93:2497-2504). As stated above, stabilizing the A2-domain (FVIII-VV, FVIII-QQ/VV) improved specific activity 2- to 4-fold over FVIII-WT when determined by one-stage clotting assay (Table 1). Specific activities were similar and consistent with commercially available B-domainless FVIII products when assayed by chromogenic. FVIII protein kinetics and clotting activity were verified across at least two individual preparations.

TABLE 1
Biochemical Characterization of FVIII Proteins. Specific activity
was determined by an aPTT based clotting assay measurement of
clotting activity relative to protein concentration. Kinetic values
were determined for FX activation by an intrinsic Xase assay using
equimolar FVIIIa and FIXa (0.25 nM) 0.25 nM FVIIIa, 0.25 nM or
0-20 nM FIXa and 0-500 nM FX in the presence of 20 μM phospholipids.
FIXa binding affinity was determined similarly using 0.25 nM FVIIIa,
0-20 nM FIXa, and 200 nM FX. Data are represented as means ±
SEM from at least two independent experiments.

| | Specific | | | |
|---|---|---|---|---|
| | Activity | Km nM | Kd nM | Vmax nM |
| | IU/mg | FX | FIXa | FXa/min |
| FVIII-WT | 9000 ± 700 | 37.6 ± 15 | 1.4 ± 0.3 | 8.7 ± 1.1 |
| FVIII-QQ | 11000 ± 900 | 50.3 ± 16 | 1.5 ± 0.3 | 10.7 ± 1.0 |
| FVIII-VV | 21000 ± 650 | 47.2 ± 22 | 1.6 ± 0.2 | 18.7 ± 2.8 |
| FVIII-QQVV | 43000 ± 950 | 51.8 ± 14 | 1.2 ± 0.3 | 21.1 ± 1.8 |

A2-domain stability was observed in FVIII species with point mutations at A2-domain interfaces Asp519 and Glu665, as both proteins kept nearly all FVIIIa activity within 15-minutes following activation. FVIII species lacking Asp519 and Glu665 mutations, FVIII-WT and FVIII-QQ, lost nearly all residual activity within the same timeframe (FIG. 2C). In stark contrast, FVIIIa-VV and FVIIIa-QQVV retained approximately 70% and 80% of their activity at 15 minutes, respectively (FIG. 2C).

In plasma, FVIII species with improved A2 domain affinity demonstrated increased peak thrombin generation across multiple FVIII concentrations compared to FVIII-WT (FIG. 2B), consistent with reported values (Wakabayashi, et al. (2009) J. Thrombosis Haemostasis 7:438-444). Additionally, FVIII-QQ and FVIII-QQVV exhibited greater thrombin generation in the presence of APC compared to FVIII-WT. FVIII and FVIII-QQ had similar thrombin generation profiles (FIG. 2B). However, FVIII-VV and FVIII-QQVV produced significantly more thrombin, while having similar profiles (FIG. 2B).

In a purified system, FVIIIa-SQ and FVIIIa-QQ displayed similar Km and Vmax values for FX activation (FIG. 2D) that were consistent with published values (Lollar, et al. (1994) J. Clin. Invest., 93 (6): 2497-2504). In contrast, the inclusion of the VV mutation caused a 2-fold increase in Vmax.

Initial thrombin generation assays were performed in the presence of an APC titration. FVIII-QQVV produced surprisingly greater amounts of thrombin compared to FVIII-SQ, FVIII-QQ, and FVIII-VV. Indeed, while FVIII-VV yielded a slight increase in thrombin generation compared to FVIII-SQ and FVIII-QQ, FVIII-QQVV yielded a surprisingly even greater increase over FVIII-VV. Indeed, the VV mutations were used for their ability to prevent A2 dissociation and are not known to affect cleavage by APC. Nonetheless, the addition of the QQ mutations to the VV mutations led to an unexpectedly superior increase in activity (synergy) in the presence of APC.

FVIIIa binding with FIXa showed a clear effect of FVIIIa stability. 5 nM FVIII was incubated with 100 nM thrombin for 30 seconds to generate FVIIIa in the presence of 0-20 nM FIXa-DEGR (enzymatically inert FIXa) and thereafter quenched with 225 nM Hirudin. 2.5 nM FVIIIa was then incubated 0-30 minutes with 0-10 nM FIXa-DEGR for 0-90% FVIIIa saturation and 80 μM PCPS with or without 6 nM APC and 50 nM PS. 0.25 nM FVIIIa was then incubated with 20 nM FIXa, 200 nM FX, and 20 μM PCPS and FVIIIa activity was then measured as a function of the rate of FXa generation.

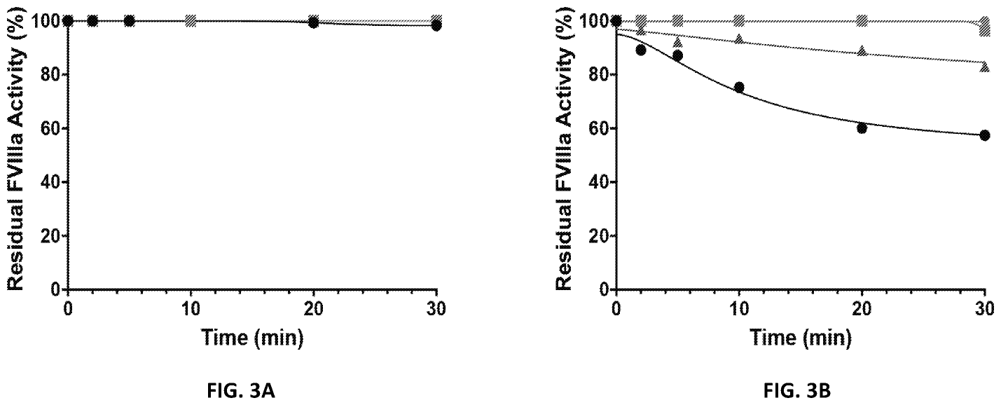

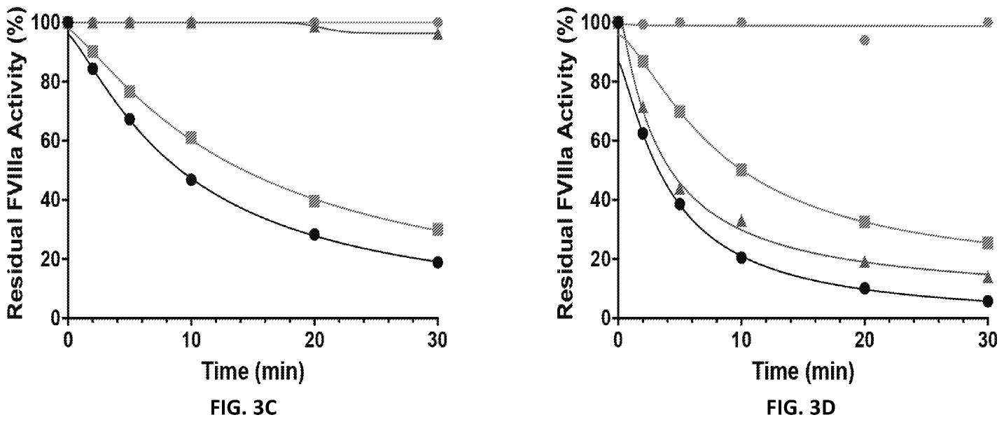

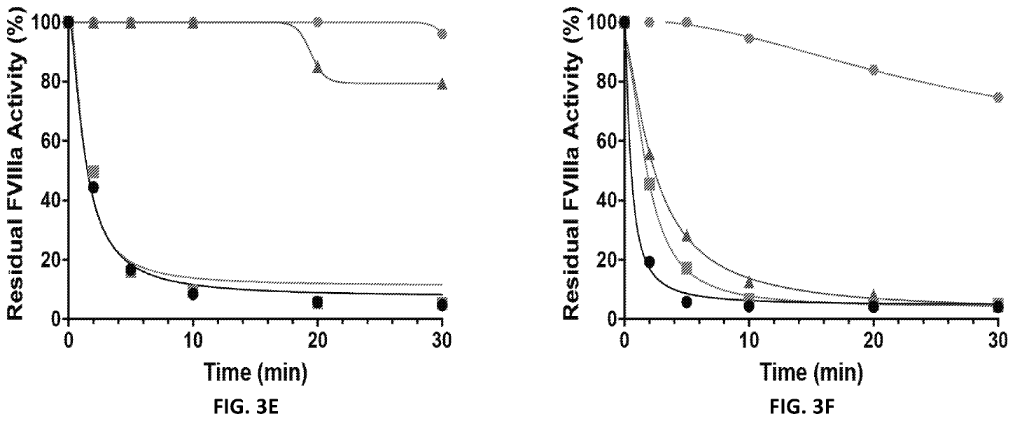

Under conditions where FVIIIa is not bound to FIXa, complete inactivation by either spontaneous A2 domain-dissociation or APC cleavage occurs within 10 minutes following activation by thrombin (FIGS. 3E and 3F, respectively). When approximately 25% of FVIIIa is bound to FIXa, ˜75% of FVIIIa-WT and FVIIIa-QQ activity is lost within 30 minutes (FIG. 3C). When APC and PS are added to the reaction, nearly all FVIIIa-WT activity is lost (FIG. 3D). Interestingly, when 25% of A2 domain-stable FVIIIa-VV is bound to FIXa, the addition of APC and PS results in a >80% reduction in residual activity. Importantly, when nearly all FVIIIa is bound to FIXa, FVIIIa is remarkably stable (FIG. 3A), while the addition of APC and PS moderately reduces residual activity in susceptible FVIII species (FIG. 3B). It is of note that, there are no observable changes in the loss of FVIIIa-QQ or FVIIIa-QQVV activity in the presence or absence of APC across varying degrees of FVIIIa-FIXa binding.

The addition of FX to the fractional saturation of FVIIIa with FIXa further increases the stability of FVIIIa. 5 nM FVIII was incubated with 100 nM thrombin for 30 seconds to generate FVIIIa in the presence of 0-20 nM FIXa-DEGR and thereafter quenched with 225 nM Hirudin. 2.5 nM FVIIIa was then incubated 0-30 minutes with 0-10 nM FIXa-DEGR for 0-90% FVIIIa saturation, 80 μM PCPS, with or without 20 nM FX-R15Q (enzymatically inert FX), with or without 6 nM APC and 50 nM PS. 0.25 nM FVIIIa was then incubated with 20 nM FIXa, 200 nM FX, and 20 μM PCPS and FVIIIa activity was then measured as a function of the rate of FXa generation.

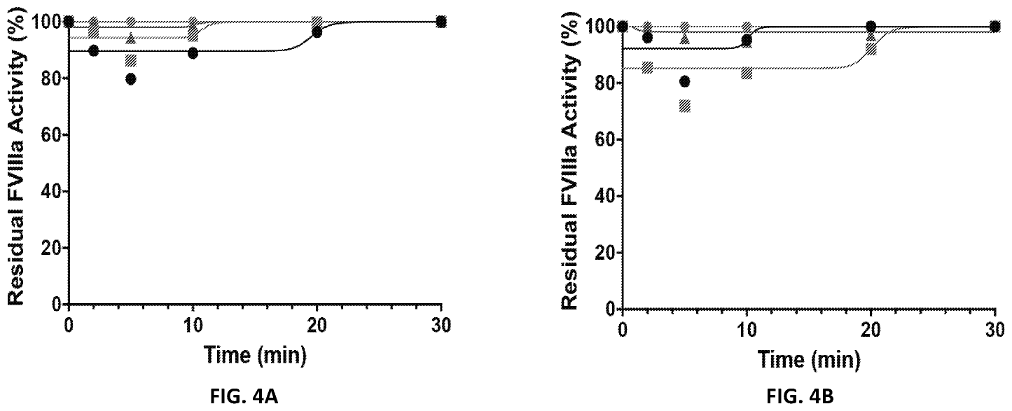

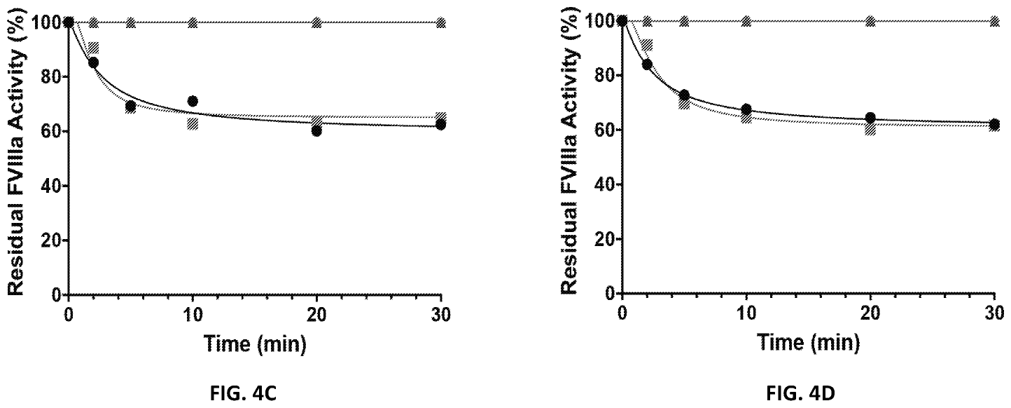

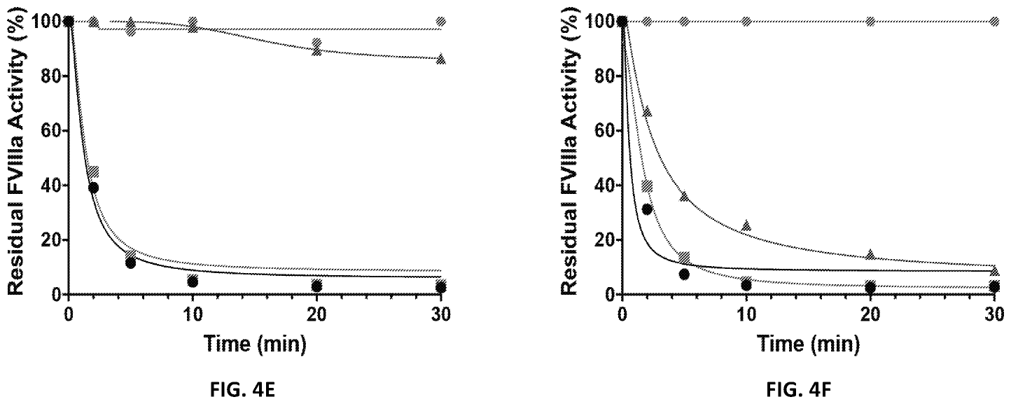

In the presence of FX, APC has no observed effect on FVIIIa-WT activity compared to a 10-40% decrease in residual activity across different levels of FVIIIa-FIXa binding when FX is not present (FIGS. 3 and 4). In APC resistant species, FVIII-QQ, the addition of FX modestly increases A2 stability under conditions where 25% of FVIIIa is bound to FIXa (FIGS. 3C and 3D and 4C and 4D). Interestingly, overall changes in residual activity in the presence of FX and APC are similar between FVIII-QQ and FVIII-WT (FIGS. 4B, 4D, and 4F). Remarkably, in the A2 domain-stable species, FVIII-VV, the addition of FX drastically reduces the effect of APC when FVIIIa is bound to FIXa (FIG. 4D). When 25% of FVIIIa-VV is bound to FIXa, there is a >80% reduction in residual activity; however, the addition of FX ameliorates the effects of APC on FVIIIa activity (FIGS. 3D and 4D). There is little to no observed loss of activity for the APC resistant and A2 domain-stable variant, FVIII-QQVV (FIG. 4).

Lastly, FVIII-QQVV has marked improved in vivo hemostatic effect, which is approximately 10-fold better in vivo than FVIII-WT in a tail clip assay.

APC displayed a role in FVIIIa inactivation at various states of FIXa binding. FVIIIa-WT and FVIIIa-VV, when exposed to APC and PS, demonstrated reduced FVIII activity compared to controls at each FIXa concentration. Further, FVIIIa-QQ and FVIIIa-VVQQ were resistant to APC inactivation, with FVIIIa-VVQQ demonstrating significant stability overall. The role of APC is most clearly seen in FVIIIa-VV, as FVIIIa inactivation by A2-dissociation is not a contributing mechanism. When FVIIIa-VV was not bound to FIXa, but was exposed to APC and PS, it lost nearly all activity within 10 minutes. As more FVIIIa-VV was bound to FIXa, more FVIIIa activity was retained, though levels were decreased overall in comparison to controls. When FVIII-VV was 100% bound to FIXa, it lost approximately 30% of its activity. An approximate 50% loss in activity in FVIII-WT is also seen. These data indicate that while FIXa confers protection against APC cleavage, it is not complete.

The stability of activated FVIII-QQVV is independent of established interactions with physiologic ligands (specifically Factor X and Factor IXa, which are components of the enzyme complex where FVIIIa has cofactor effect). The stability of FVIIIa-QQVV or protection from A2-domain dissociation is independent of FIXa interactions and stability of FVIIIa-QQVV from APC cleavage is independent of FX.

The specific activity of FVIII-QQVV is unexpectedly superior. Specifically, the specific activity of FVIII-QQVV is surprisingly higher than FVIII-VV. This unexpected increase in specific activity indicates synergy between combing the QQ mutations (APC resistance mutations that do not impact specific activity of the protein) with VV mutations (stabilizing the A2 domain, which does improve specific activity but only ˜2-fold).

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A Factor VIII (FVIII) variant comprising a substitution mutation of the Arg at position 336; the Arg at position 562; the Asp at position 519; and the Glu at position 665.

2. The FVIII variant of claim 1, wherein the Arg at position 336 is substituted with Gln.

3. The FVIII variant of claim 1, wherein the Arg at position 562 is substituted with Gln.

4. The FVIII variant of claim 1, wherein the Asp at position 519 is substituted with Val.

5. The FVIII variant of claim 1, wherein the Glu at position 665 is substituted with Val.

6. The FVIII variant of claim 1, wherein the Arg at position 336 is substituted with Gln, the Arg at position 562 is substituted with Gln, the Asp at position 519 is substituted with Val, and the Glu at position 665 is substituted with Val.

7. The FVIII variant of claim 1, wherein the variant lacks the B domain or the B domain has been replaced by a peptide linker.

8. The FVIII variant of claim 1, wherein said FVIII comprises amino acids 1-740 and 1649-2332 of SEQ ID NO: 1.

9. The FVIII variant of claim 1, wherein said FVIII comprises amino acids 1-740 and 1690-2332 of SEQ ID NO: 1.

10. A composition comprising at least one FVIII variant of claim 1 and at least one pharmaceutically acceptable carrier.

11. A method for treatment of a hemostasis related disorder in a patient in need thereof comprising administration of a therapeutically effective amount of the FVIII variant of claim 1 in a pharmaceutically acceptable carrier.

12. The method of claim 11, wherein said hemostasis related disorder is hemophilia.

13. An isolated nucleic acid molecule encoding the FVIII variant of claim 1.

14. The nucleic acid molecule of claim 13, wherein said FVIII variant comprises a signal peptide.

15. An expression vector comprising the nucleic acid molecule of claim 13 operably linked to a regulatory sequence.

16. The vector of claim 15, selected from the group consisting of a non-viral vector, an adenoviral vector, an adenovirus-associated vector, a retroviral vector, a plasmid, and a lentiviral vector.

17. A host cell comprising the vector of claim 16.

18. The host cell of claim 17, wherein said host cells are human cells.

19. A method for treatment of a hemostasis related disorder in a subject in need thereof comprising administration of a therapeutically effective amount of the vector of claim 15 in a pharmaceutically acceptable carrier to the subject.

20. The method of claim 19, wherein said hemostasis related disorder is hemophilia.

21. The activated form of the FVIII variant of claim 1.

22. A method for reducing blood loss in a patient in need thereof comprising administration of a therapeutically effective amount of the FVIII variant of claim 1 in a pharmaceutically acceptable carrier.

23. A method for treatment of a hemostasis related disorder in a subject in need thereof comprising editing the FVIII gene of the subject to comprise a sequence encoding a substitution mutation of the Arg at position 336; the Arg at position 562; the Asp at position 519; and the Glu at position 665.

24. A method for reducing blood loss in a patient in need thereof comprising administration of a therapeutically effective amount of the nucleic acid molecule of claim 13 in a pharmaceutically acceptable carrier.
